SEQUENCE LISTING (7 sequences)

SEQ ID NO: 1 - protein, 35 aa, artificial
  synthetic
  DOMAIN (1)..(35): Module C (ADF-4)
GSSAAAAAAAASGPGGYGPENQGPSGPGGYGPGGP

SEQ ID NO: 2 - protein, 35 aa, artificial
  synthetic
  DOMAIN (1)..(35): Module Cc
GSSAAAAAAAASGPGGYGPENQGPCGPGGYGPGGP

SEQ ID NO: 3 - protein, 560 aa, artificial
  synthetic
  REPEAT (1)..(560): C16
GSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGP

SEQ ID NO: 4 - protein, 1120 aa, artificial
  synthetic
  REPEAT (1)..(1120): C32
GSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGP

SEQ ID NO: 5 - protein, 1680 aa, artificial
  synthetic
  REPEAT (1)..(1680): C48
GSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGP

SEQ ID NO: 6 - protein, 280 aa, artificial
  synthetic
  REPEAT (1)..(280): C8
GSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGP

SEQ ID NO: 7 - protein, 40 aa, Artificial
  synthetic
  Domain (1)..(40): Module Clys
GRGSMGSSAAAAAAAASGPGGYGPKNQGPSGPGGYGPGGP

(19) United States
(12) Patent Application Publication
(10) Pub. No.: US 2024/0301015 A1
(43) Pub. Date: Sep. 12, 2024

(54) SILK POLYPEPTIDE FORMULATION COMPRISING FORMATE

(71) Applicant: AMSILK GMBH, Planegg/Martinsried (DE)

(72) Inventors: Jens Klein, Pullach I. Isartal (DE); Lin Romer, Ottobrunn (DE); Andreas Schmideder, Velden (DE)

(21) Appl. No.: 18/568,183

(22) PCT Filed: Jun. 2, 2022
(86) PCT No.: PCT/EP2022/065144

§ 371 Date: Dec. 7, 2023

(30) Foreign Application Priority Data
Jun. 10, 2021 (EP) 21178894.8

(51) Int. Cl. C07K 14/435

(52) U.S. Cl. CPC C07K 14/43518

(57) ABSTRACT

The present invention relates to an acidic formulation comprising a silk polypeptide and formate. The present invention further relates to a method for producing said formulation. The present invention also relates to the uses of said formulation.

The present invention relates to an acidic formulation comprising a silk polypeptide and formate. The present invention further relates to a method for producing said formulation. The present invention also relates to the uses of said formulation.

### BACKGROUND OF THE INVENTION

Silk polypeptides represent a unique family of structural molecules. The molecular structure of many silks consists of large regions or domains of hydrophobic amino acids, segregated by relatively short and more hydrophilic regions. Silk polypeptides exhibit superior mechanical properties. They are promising materials for drug delivery and tissue engineering due to their biocompatibility, biodegradability, self-assembly, and controllable structure and morphology. Additionally, silk materials exhibit high encapsulation efficiency and controllable drug release kinetics due to the adjustment of crystalline β-sheet formation. The formation of silk polypeptide biomaterials, such as fibers, films, mats, scaffolds, or capsules, is well known in the art. Moreover, silk polypeptides are well known in the cosmetic field, e.g. as cosmetic ingredient.

However, the known silk polypeptide solutions have the disadvantage that the silk polypeptides are not homogenously distributed at high concentrations. Silk polypeptide precipitation at high concentrations is, however, detrimental to the quality of the resulting silk products.

Thus, there is still a need for new silk polypeptide formulations which can be used in many applications such as in the agrochemical industry, cleaning industry, home care industry, detergent industry, cosmetic industry, or food industry. These new silk polypeptide formulations should particularly comprise silk polypeptides at high concentrations. It would especially be advantageous for many applications, e.g. in the agrochemical industry, cleaning industry, home care industry, detergent industry, cosmetic industry, or food industry, to use liquid or flowable formulations which are easy to apply. These liquid or flowable formulations should comprise the highest possible concentrations of silk polypeptides to prevent dilution of the other active substances and to minimize the environmental footprint for transportation as well as the volumes to be used by the user. In addition, the production costs for silk polypeptide formulations should be significantly reduced in order to ensure a sustainable market entry, in particular in application areas with lower added value.

The present inventors were surprisingly be able to provide silk polypeptide formulations comprising a silk polypeptide and formate. In addition, the present inventors were surprisingly be able to provide a new production process for the generation of silk polypeptide formulations comprising a silk polypeptide and formate. These formulations encompass silk polypeptides at high concentrations. Said formulations can be used in different fields, e.g. in the agrochemical industry, cleaning industry, home care industry, detergent industry, cosmetic industry, or food industry. In particular, the presence of fibrillary structures comprised in the silk polypeptide formulations simplifies and improves the coating of substrates with said formulations.

### SUMMARY OF THE INVENTION

In a first aspect, the present invention relates to an (flowable or non-flowable/solid) acidic formulation comprising a silk polypeptide and formate, wherein the concentration of the silk polypeptide in the formulation is in the range of 0.25% by weight to 45% by weight. The silk polypeptide is preferably comprised in the formulation in an aggregated form.

In a second aspect, the present invention relates to a method for producing a(n) (flowable or non-flowable/solid) acidic formulation comprising the steps of:
(i) providing an acidic solution comprising a silk polypeptide and formic acid, wherein the concentration of the silk polypeptide in the solution is in the range of 0.5% by weight to 45% by weight, and
(ii) forming a(n) (flowable or non-flowable/solid) acidic formulation from the solution. The silk polypeptide is preferably comprised in the formulation in an aggregated form.

In a third aspect, the present invention relates to the use of the (flowable or non-flowable/solid) acidic formulation of the first aspect for silk polypeptide storage.

In a fourth aspect, the present invention relates to the use of the (flowable or non-flowable/solid) acidic formulation of the first aspect in the agrochemical industry, cleaning industry, home care industry, detergent industry, cosmetic industry, or food industry.

In a fifth aspect, the present invention relates to a substrate comprising the (flowable or non-flowable/solid) acidic formulation of the first aspect.

In a sixth aspect, the present invention relates to a composition comprising the (flowable or non-flowable/solid) acidic formulation of the first aspect.

This summary of the invention does not necessarily describe all features of the present invention. Other embodiments will become apparent from a review of the ensuing detailed description.

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B, and Kölbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety. Nothing herein is to be construed as an admission that the invention is not entitled to antedate such disclosure by virtue of prior invention.

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

The term “comprise” or variations such as “comprises” or “comprising” according to the present invention means the inclusion of a stated integer or group of integers but not the exclusion of any other integer or group of integers. The term “consisting essentially of” according to the present invention means the inclusion of a stated integer or group of integers, while excluding modifications or other integers which would materially affect or alter the stated integer. The term “consisting of” or variations such as “consists of” according to the present invention means the inclusion of a stated integer or group of integers and the exclusion of any other integer or group of integers.

The terms “a” and “an” and “the” and similar reference used in the context of describing the invention (especially in the context of the claims) are to be construed to cover both the singular and the plural, unless otherwise indicated herein or clearly contradicted by context.

The terms “polypeptide” and “protein” are used interchangeably in the context of the present invention. They refer to a long peptide-linked chain of amino acids, e.g. one that is at least 20 amino acids long.

The term “silk polypeptide”, as used herein, refers to a polypeptide which shows, in comparison to other polypeptides, a quite aberrant amino acid composition. In particular, a silk polypeptide possesses large quantities of hydrophobic amino acids such as glycine or alanine. In addition, a silk polypeptide contains highly repetitive amino acid sequences or repetitive units (repeat units, modules), especially in their large core domain. Based on DNA analysis, it was shown that all silk polypeptide are chains of repetitive units which further comprise a limited set of distinct shorter peptide motifs. The expressions “peptide motif” and “consensus sequence” can be used interchangeably herein. Generally, the silk consensus sequences can be grouped into four major categories: GPGXX, GGX, Aₓ or (GA)ₙ and spacers. These categories of peptide motifs in silk proteins have been assigned structural roles. For example, it has been suggested that the GPGXX motif is involved in a β-turn spiral, probably providing elasticity. The GGX motif is known to be responsible for a glycine-rich 3₁-helix. Both GPGXX and GGX motifs are thought to be involved in the formation of an amorphous matrix that connects crystalline regions, thereby providing elasticity of the fiber. Alanine-rich motifs typically contain 6-9 residues and have been found to form crystalline β-sheets. The spacers typically contain charged groups and separate the iterated peptide motifs into clusters. The silk polypeptide can perform self-assembly. Preferably, the silk polypeptide is a spider silk polypeptide. More preferably, the silk polypeptide, e.g. spider silk polypeptide, is a recombinant polypeptide.

The term “self-assembly”, as used herein, refers to a process in which a disordered system of pre-existing polypeptides forms an organized structure or pattern as a consequence of specific, local interactions (e.g. van der Waals forces, hydrophobic interactions, hydrogen bonds, and/or salt-bridges, etc.) among the polypeptides themselves, without external direction or trigger although external factors might influence speed and nature of self-assembly. This particularly means that when two or more disordered and/or unfolded polypeptides are brought into contact, they interact with each other and consequently form a three-dimensional structure. This three-dimensional structure can also be considered as a polypeptide aggregate. The change from a disordered system to an organized structure or pattern during self-assembly is characterized by a transition from a fluid state to a gelatinous/gel-like and/or solid state and a corresponding increase in viscosity. The transition from a fluid state to a gelatinous/gel-like and/or solid state can be monitored, for example, by optical measurement or rheology. These techniques are known to the skilled person. The transition from a fluid state to a gelatinous/gel-like and/or solid state can be monitored, for example, using optical methods. As mentioned above, polypeptide aggregates are formed during the process of self-assembly.

The term “polypeptide aggregates”, as used herein, refers to polypeptide structures which are formed as a consequence or result of polypeptide self-assembly. In the process of polypeptide self-assembly, multiple copies/units of polypeptides self-aggregate into a body or mass without external direction or trigger although external factors might influence speed and nature of self-assembly. In the polypeptide aggregates, the different polypeptides are connected with or attached to each other via covalent (e.g. disulfide bridges) and/or non-covalent interactions (e.g. van der Waals forces, hydrophobic interactions, hydrogen bonds, and/or salt-bridges). It should be clear that a polypeptide aggregate encompasses at least two polypeptides.

In the context of the present invention, the self-assembly of silk polypeptides is described. During the process of silk polypeptide self-assembly, silk polypeptide aggregates are formed.

The term “acidic solution”, as used wherein, refers to any solution in which an acid is the solvent. In the context of the present invention, the acidic solution encompasses formic acid and a silk polypeptide. In a particular embodiment, the acidic solution is an aqueous acidic solution which encompasses, in addition to formic acid and a silk polypeptide, water. The silk polypeptide is comprised in the acidic solution of the present invention in solubilized form. The acidic solution differs from the acidic formulation (as described below) in that it has a viscosity which is usually lower than the viscosity of the acidic formulation. In particular, the acidic solution does not comprise a silk polypeptide having a fibrillary structure.

The term “acidic formulation”, as used herein, refers to a formulation comprising a silk polypeptide and formate. In the acidic formulation, as described herein, the silk polypeptide is homogenously distributed. Preferably, the concentration of the silk polypeptide in the acidic formulation is in the range of 0.25% by weight to 45% by weight. The acidic formulation, as described herein, has preferably a clear appearance and/or does not comprise visible precipitates. Said visible precipitates are usually the cause for clouding. In the context of the present invention, the acidic formulation encompasses formate and a silk polypeptide. In a particular embodiment, the acidic formulation is an aqueous acidic formulation which encompasses, in addition to formate and a silk polypeptide, water. In particular, the term “acidic formulation” refers to a formulation comprising fibrillary complexes of silk polypeptides. In said fibrillary complexes, the silk polypeptides are oriented and/or conjoined to each other. Said fibrillary complexes of silk polypeptides may be formed by self-assembly of the silk polypeptides in the acidic solution. The mechanism of silk polypeptide self-assembly may include covalent and/or non-covalent interactions between the silk polypeptides. The presence of fibrillary complexes in the acidic formulation improves and simplifies the coating of substrates with said acidic formulation. The fibrillary complexes may have the form of net structures.

In a preferred embodiment, the silk polypeptide is comprised in the acidic formulation in an aggregated form. Silk polypeptide aggregates are the result of silk polypeptide self-assembly. As mentioned above, the acidic formulation of the present invention has preferably a clear appearance and/or does not comprise visible precipitates. In particular, the acidic formulation, as described herein, comprises fibrillary complexes of silk polypeptides. In contrast thereto, a turbid acidic formulation comprises visible precipitates. The silk polypeptides comprised therein particularly show a diffuse and unoriented aggregation. They have mainly a random orientation and are not fibrillary.

Especially, the acidic formulation of the present invention is flowable or non-flowable. The non-flowable acidic formulation can also be designated as solid acidic formulation.

A formulation after pH increase to neutralization may not be designated as acidic anymore. The acidic formulation after neutralization may simply be designated as formulation which comprises water, i.e. an aqueous formulation.

The term “flowable acidic formulation”, as used herein, refers to a formulation that is able/capable of flowing or being flowed. A flowable acidic formulation is (still) in a liquid state. Preferably, the concentration of the silk polypeptide in the flowable acidic formulation is in the range of 0.25% by weight to 4% by weight. In a particular embodiment, the flowable acidic formulation is a flowable aqueous acidic formulation which encompasses, in addition to formate and a silk polypeptide, water.

The term “non-flowable acidic formulation”, as used herein, refers to a formulation that is not able/capable of flowing or being flowed (anymore). A non-flowable acidic formulation is not in a liquid state anymore. It can, thus, also be designated as solid. Preferably, the concentration of the silk polypeptide in the non-flowable/solid acidic formulation is in the range of >4% by weight to 45% by weight. In a particular embodiment, the non-flowable acidic formulation is a non-flowable aqueous acidic formulation which encompasses, in addition to formate and a silk polypeptide, water.

The followability or non-followability of an acidic formulation can easily be determined by the skilled person, e.g. by rheology or viscosity measurements. The followability or non-followability measurements are preferably preformed under standard conditions (25° C.).

As mentioned above, the silk polypeptide is comprised in the acidic formulation in an aggregated form. In this respect, it should be noted that the percentage of aggregated silk polypeptide in the non-flowable/solid acidic formulation is higher than the percentage of aggregated silk polypeptide in the flowable acidic formulation.

In particular, the flowable or non-flowable acidic formulation, as described herein, has a fibrillary structures or comprises fibrillary complexes. As mentioned above, the presence of fibrillary complexes improves and simplifies the coating of substrates with said acidic formulation. The fibrillary complexes may have the form of net structures.

The acidic formulation has preferably a gel-like structure, e.g. a flowable or non-flowable/solid gel-like structure. It is more preferably a hydrogel, e.g. a flowable or non-flowable/solid hydrogel.

The acidic formulations described herein are preferably stable acidic formulations. Thus, they preferably do not change their shape and/or texture over a time period of 0.5, 1, 2, 3, 4, 5, 6, 7, 8, 9, 10, 11, 12, 13, 14, 15, 16, 17, 18, 19, 20, 21, 22, 23 hours, 1, 2, 3, 4, 5, 6 days, 1, 2, 3 weeks, 1, 2, 3, 4, 5, 6, 7, 8, 9, 10, 11 months, 1, 2, 3, 4, or 5 years.

The term “hydrogel”, as used herein, refers to a structure that is formed if the concentration of silk polypeptides is high enough to build a continuous polymeric network of silk polypeptides by which the liquid component is constrained in its mobility. In particular, the hydrophilic polymeric network of silk polypeptides is formed by silk polypeptide self-assembly. It is further stabilized by chemical and/or physical interactions between the silk polypeptides. The network is dispersed throughout a phase limited in its mobility. The hydrophilicity and stability of the hydrogel permits the penetration and absorption of water (swelling) without dissolving, thus, maintaining its three-dimensional (3D) structure and function. The hydrogel is an excellent material candidate for a variety of biological applications. These applications include, but are not limited to, agrochemical industry, cleaning industry, home care industry, detergent industry, or cosmetic industry, or food industry. Preferably, the concentration of the silk polypeptide in the hydrogel is in the range of 0.25% by weight to 45% by weight. In particular, the hydrogel of the present invention is flowable or non-flowable. The non-flowable hydrogel can also be designated as solid hydrogel.

The term “flowable hydrogel”, as used herein, refers to a hydrogel that is able/capable of flowing or being flowed. A flowable hydrogel is (still) in a liquid state. Preferably, the concentration of the silk polypeptide in the flowable hydrogel is in the range of 0.25% by weight to 4% by weight.

The term “non-flowable hydrogel”, as used herein, refers to a hydrogel that is not able/capable of flowing or being flowed (anymore). A non-flowable hydrogel is not in a liquid state anymore. It can, thus, also be designated as solid.

Preferably, the concentration of the silk polypeptide in the non-flowable/solid hydrogel is in the range of >4% by weight to 45% by weight.

The followability or non-followability of a hydrogel can easily be determined by the skilled person, e.g. by rheology or viscosity measurements. The followability or non-followability measurements are preferably preformed under standard conditions (25° C.).

In particular, the flowable or non-flowable hydrogel, as described herein, has a fibrillary structures or comprises fibrillary complexes. As mentioned above, the presence of fibrillary complexes improves and simplifies the coating of substrates with said acidic formulation. The fibrillary complexes may have the form of net structures.

The term “compound”, as used herein, refers to any compound having a purpose that may be useful in the present invention, e.g. a compound useful in the agrochemical industry, cleaning industry, home care industry, detergent industry, cosmetic industry, or food industry. The compound is preferably part of the acidic formulation described herein. Acidic formulations comprising the compound may be formulated by mixing the compound with the acidic solution used to make the acidic formulation. Alternatively, the compound can be coated onto, attached to, or incorporated in the acidic formulation after its formation. The compound may be present as a liquid, a finely divided solid or in any other appropriate physical form. The compound useful in the agrochemical industry may be selected from the group consisting of dyestuff, odoriferous substance, sunscreen, fertilizer, pesticide, hormone, growth factor, insecticide, pesticide, herbicide, fungizide, microorganisms, and nutrient. The compound useful in the food industry may be selected from the group consisting of a nutrient, vitamin, aroma, preservative and dye. The compound useful in the cleaning industry and homecare industry may be selected from the group consisting of dyestuff, antimicrobial substance, antiviral substance, detergent, softener, surfactant, enzyme, fragrance, alcohol, greying inhibitor, dye transfer inhibitor, metal complex, nitrile, inorganic compound, bleaching agent, tenside, fatty acid, carbonic acid, silicate, carbonate, polymer, silicone, and brightening agent.

The acidic formulation described herein may be applied as a coating to a substrate.

The term “coating”, as used herein, refers to a covering that is applied to the substrate, in particular to the surface of the substrate, to be coated. The coating itself may be an all-over coating, completely covering the substrate, or it may only cover parts of the substrate. In one embodiment, the coating covers at least 1%, preferably at least 30%, more preferably at least 50%, even more preferably at least 80%, and most preferably at least 90% or even 100% of the surface of the substrate. In one preferred embodiment, the coating is an uniform and/or homogenous coating. It has preferably a thickness of between 10 nm and 1 mm and more preferably a thickness of between 50 nm and 0.5 μm.

The coating has preferably the form of a hydrogel. It is preferably achieved by dip coating and/or spray coating.

In an example, the substrate is a plant. In this case, the coating may be applied to (parts of) the root(s) of the plant and/or sprout(s) of the plant/aboveground part(s) of the plant. The coating may also be applied to (parts of) the fruits and/or blossoms/flower petals of the plant. It should be clear that a plant, in particular (a/an) sprout(s) of a plant/aboveground part(s) of a plant, also comprise(s) leaves/leafage. Thus, the coating preferably also covers the leaves/leafage. The coating is preferably applied pre-harvest, e.g. to a seedling, a growing plant, or full-grown plant. Said substrate may be selected from the group consisting of a plant, a part of a plant (e.g. root and/or sprout), plant seeds, a nutrient, a fruit, and a flower.

The acidic formulation described herein may alternatively be part of a composition. The composition may be, for example, a composition useful in the agrochemical industry, cleaning industry, home care industry, detergent industry, cosmetic industry, or food industry. Such composition may be a food composition, a cleaning composition, a fertilizer composition, or a plant or seed protecting composition. In these cases, the acidic formulation may be integrated, embedded, or worked into the composition.

The term “non-covalent linkage” means a type of linkage (interaction) that does not involve the sharing of pairs of electrons, but rather involves more dispersed variations of electromagnetic interactions, ionic (electrostatic) interactions, hydrophobic interactions, and/or van der Waals interactions.

The term “isoelectric point (pI) of a polypeptide”, as used herein, refers to a value which represents the pH at which the polypeptide is electrically neutral or carries no net electrical charge. It is a physicochemical property resulting from ambivalent charge properties of its amino acid constituents. The pH of surrounding environment can affect the net charge of the polypeptide through the gain or loss of protons and make the polypeptide more positively or negatively charged. In acidic solution, the polypeptide is positively charged and migrate towards the cathode. In basic solution, the polypeptide is negatively charged and migrates towards anode. The pH at which protein molecule exists as a neutral dipolar ion and not migrate to either electrode is the pI. Techniques to determine the pI of a polypeptide are well known in the art. These techniques include, for example, flat gel isoelectric focusing or capillary isoelectric focusing. It is also possible to determine the pI by mathematical means on the basis of the amino acid sequence of the polypeptide. There are a number of internet platforms available allowing the mathematical calculation of the pI of a polypeptide on the basis of its amino acid sequence. Here, it is exemplary referred to the Expasy Bioinformatic Resource Portal.

### EMBODIMENTS OF THE INVENTION

As mentioned above, there is still a need for new silk polypeptide formulations which can be used in many applications such as in the agrochemical industry, cleaning industry, home care industry, detergent industry, cosmetic industry, or food industry. The new silk polypeptide formulations should particularly comprise silk polypeptides at high concentrations. The present inventors were surprisingly be able to provide silk polypeptide formulations having these properties.

Thus, in a first aspect, the present invention relates to an acidic formulation comprising a silk polypeptide and formate, wherein the concentration of the silk polypeptide in the formulation is in the range of 0.25% by weight to 45% by weight. Formate is the salt of formic acid. It is produced by increasing the pH, e.g. to neutralization. In particular, the acidic formulation is an aqueous acidic formulation. The aqueous acidic formulation comprises, in addition to a silk polypeptide and formate, water.

Preferably, the concentration of the silk polypeptide in this formulation is in the range of 0.5% by weight to 40% by weight, more preferably in the range of 5% by weight to 30% by weight, even more preferably in the range of 5% by weight to 25% by weight, and most preferably in the range of 5% by weight to 20% by weight.

It is particularly preferred that the concentration of the silk polypeptide in this formulation is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. It is more particularly preferred that the concentration of the silk polypeptide in this formulation is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

It is preferred that the silk polypeptide is comprised in the acidic formulation in an aggregated form. The silk polypeptide aggregation is the result of silk polypeptide self-assembly.

Accordingly, in one preferred embodiment, the present invention relates to an acidic formulation comprising a silk polypeptide and formate, wherein the concentration of the silk polypeptide in the formulation is in the range of 0.25% by weight to 45% by weight, and wherein the silk polypeptide is comprised in the formulation in an aggregated form.

Preferably, the concentration of the silk polypeptide in this formulation is in the range of 0.5% by weight to 40% by weight, more preferably in the range of 5% by weight to 30% by weight, even more preferably in the range of 5% by weight to 25% by weight, and most preferably in the range of 5% by weight to 20% by weight. It is particularly preferred that the concentration of the silk polypeptide in this formulation is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. It is more particularly preferred that the concentration of the silk polypeptide in this formulation is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

It is (additionally or alternatively) further preferred that the silk polypeptide comprised in the acidic formulation has a fibrillary structure.

Accordingly, in one further preferred embodiment, the present invention relates to an acidic formulation comprising a silk polypeptide and formate, wherein the concentration of the silk polypeptide in the formulation is in the range of 0.25% by weight to 45% by weight, and wherein the silk polypeptide is comprised in the formulation in an aggregated form and has a fibrillary structure.

Preferably, the concentration of the silk polypeptide in this formulation is in the range of 0.5% by weight to 40% by weight, more preferably in the range of 5% by weight to 30% by weight, even more preferably in the range of 5% by weight to 25% by weight, and most preferably in the range of 5% by weight to 20% by weight.

It is particularly preferred that the concentration of the silk polypeptide in this formulation is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. It is more particularly preferred that the concentration of the silk polypeptide in this formulation is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

It is (additionally or alternatively) still further preferred that the acidic formulation has a pH which is equal or higher than the pI of the silk polypeptide. In case of the silk polypeptide C₁₆, as an example, this means the following: The silk polypeptide C₁₆ has a pI of 3.48. Thus, the acidic formulation comprising the silk polypeptide C₁₆ has preferably a pH which is equal or higher than 3.48. In particular, the pH is at least 0.1 pH units higher than the pI of the silk polypeptide. Preferably, the pH is at least 0.2, 0.3, 0.4, 0.5, 0.6, 0.7, 0.8, 0.9, 1, 2, 3, 4, 5, 6, 7, or 8 pH units higher than the pI of the silk polypeptide. In case of the silk polypeptide C₁₆, as an example, this means the following: The silk polypeptide C₁₆ has a pI of 3.48. Thus, the acidic formulation comprising the silk polypeptide C₁₆ has preferably a pH which is at least 3.58, at least 3.68, at least 3.78, at least 3.88, at least 3.98, at least 4.08, at least 4.18, at least 4.28, at least 4.38, at least 4.48, at least 5.48, at least 6.48, at least 7.48, at least 8.48, or at least 9.48. More preferably, the acidic formulation comprising the silk polypeptide C₁₆ has a pH which is between 3.5 and 5. In one alternative, the acidic formulation comprising the silk polypeptide C₁₆ has a pH which is between 5 and 7. In one another alternative, the acidic formulation comprising the silk polypeptide C₁₆ has a pH which is between 7 and 10.

Accordingly, in one more preferred embodiment, the present invention relates to an acidic formulation comprising a silk polypeptide and formate, wherein the concentration of the silk polypeptide in the formulation is in the range of 0.25% by weight to 45% by weight, wherein the formulation has a pH which is equal or higher than the pI of the silk polypeptide, and wherein the silk polypeptide is comprised in the formulation in an aggregated form and has a fibrillary structure. In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

Preferably, the concentration of the silk polypeptide in this formulation is in the range of 0.5% by weight to 40% by weight, more preferably in the range of 5% by weight to 30% by weight, even more preferably in the range of 5% by weight to 25% by weight, and most preferably in the range of 5% by weight to 20% by weight.

It is particularly preferred that the concentration of the silk polypeptide in this formulation is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. It is more particularly preferred that the concentration of the silk polypeptide in this formulation is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

It is (alternatively or additionally) also preferred that the silk polypeptide is a recombinant silk polypeptide. The (recombinant) silk polypeptide may be a spider silk polypeptide, e.g. a major ampullate silk polypeptide such as a dragline silk polypeptide, a minor ampullate silk polypeptide, or a flagelliform silk polypeptide of an orb-web spider. Particularly, the silk polypeptide is a spider silk polypeptide. More particularly, the spider silk polypeptide is a recombinant spider silk polypeptide.

It is particularly preferred that the (recombinant) silk polypeptide is a polypeptide with an amino acid sequence which comprises or consists of at least 50%, 60%, 65%, 70%, 75%, 80%, 85%, or 90% multiple copies of repetitive units. It is more preferred that the silk polypeptide is a polypeptide with an amino acid sequence which comprises or consists of at least 95% multiple copies of repetitive units. Said repetitive units may be identical or different.

It is further particularly preferred that the (recombinant) silk polypeptide consists of between 40 to 4000 amino acids. It is more preferred that the (recombinant) silk polypeptide consists of between 100 to 3500 amino acids or between 200 to 2500 amino acids. It is even more preferred that the (recombinant) silk polypeptide consists of between 250 to 2000 amino acids.

It is also particularly preferred that the (recombinant) silk polypeptide comprises at least two identical repetitive units. For example, the (recombinant) silk polypeptide may comprise between 2 to 100 repetitive units, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 repetitive units.

It is particularly more preferred that the repetitive units are independently selected from the group consisting of module C (SEQ ID NO: 1) or variants thereof, module C^{Cys} (SEQ ID NO: 2) or variants thereof, and module C^{Lys} (SEQ ID NO: 7) or variants thereof. Module C^{Cys} (SEQ ID NO: 2) is a variant of module C (SEQ ID NO: 1). In this module, the amino acid Ser at position 25 has been replaced by the amino acid Cys. Module C^{Lys} (SEQ ID NO: 7) is also a variant of module C (SEQ ID NO: 1). Module C^{Cys} can also be designated as module C^{C}.

The module C variant differs from the reference module C from which it is derived by up to 1, 2, 3, 4, 5, 6, 7, 8, 9, or 10, 11, 12, 13, 14, or 15 amino acid changes in the amino acid sequence (i.e. substitutions, additions, insertions, deletions, N-terminal truncations and/or C-terminal truncations). Such a module variant can alternatively or additionally be characterized by a certain degree of sequence identity to the reference module from which it is derived. Thus, the module C variant has a sequence identity of at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or even 99.9% to the respective reference module C. Preferably, the sequence identity is over a continuous stretch of at least 5, 10, 15, 18, 20, 24, 27, 28, 30, 34, or more amino acids, preferably over the whole length of the respective reference module C.

The sequence identity may be at least 80% over the whole length, may be at least 85% over the whole length, may be at least 90% over the whole length, may be at least 95% over the whole length, may be at least 98% over the whole length, or may be at least 99% over the whole length of the respective reference module C. Alternatively, the sequence identity may be at least 80% over a continuous stretch of at least 5, 10, 15, 18, 20, 24, 28, or 30 amino acids, may be at least 85% over a continuous stretch of at least 5, 10, 15, 18, 20, 24, 28, or 30 amino acids, may be at least 90% over a continuous stretch of at least 5, 10, 15, 18, 20, 24, 28, or 30 amino acids, may be at least 95% over a continuous stretch of at least 5, 10, 15, 18, 20, 24, 28, or 30 amino acids, may be at least 98% over a continuous stretch of at least 5, 10, 15, 18, 20, 24, 28, or 30 amino acids, or may be at least 99% over a continuous stretch of at least 5, 10, 15, 18, 20, 24, 28, or 30 amino acids of the respective reference module C.

A fragment (or deletion) variant of module C has preferably a deletion of up to 1, 2, 3, 4, 5, 6, 7, 8, 9, 10, 11, 12, 13, 14, or 15 amino acids at its N-terminus and/or at its C-terminus. The deletion can also be internally.

Additionally, the module C variant or fragment is only regarded as a module C variant or fragment within the context of the present invention, if the modifications with respect to the amino acid sequence on which the variant or fragment is based do not negatively affect the ability of the silk polypeptide to be solubilized (without the formation of precipitate) in an acidic solution comprising formic acid. The skilled person can readily assess whether the silk polypeptide comprising a module C variant or fragment still has this property. In this respect, it is referred to the examples comprised in the experimental part of the present patent application. Module C^{Cys} or C^{Lys} variants may also be encompassed by the present invention. Regarding the module C^{Cys} or C^{Lys} variants, the same explanations/definitions apply which have been made with respect to the module C variant (see above).

It is particularly even more preferred that the silk polypeptide is selected from the group consisting of (C)ₘ, (C^{Cys})ₘ, (C^{Lys})ₘ, (C)ₘC^{Cys}, C^{Cys}(C)ₘ, C^{Lys}(C)ₘ, and (C)ₘC^{Lys}, wherein m is an integer of 2 to 96, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96.

It is particularly most preferred that the silk polypeptide is selected from the group consisting of C^{Lys}C₁₆, C^{Lys}C₃₂, C^{Lys}C₄₈, C₁₆C^{Lys}, C₃₂C^{Lys}, C₄₈C^{Lys}, C₁₆, C₃₂, C₄₈, C^{Cys}C₁₆, C^{Cys}C₃₂, C^{Cys}C₈, C₁₆C^{Cys}, C₃₂C^{Cys}, and C₄₈C^{Cys}.

The silk polypeptide C₁₆ (16 times module C) has the amino acid sequence according to SEQ ID NO: 3, the silk polypeptide C₃₂ (32 times module C) has the amino acid sequence according to SEQ ID NO: 4, the silk polypeptide C₄₈ (48 times module C) has the amino acid sequence according to SEQ ID NO: 5, and the silk polypeptide C₈ has the amino acid sequence according to SEQ ID NO: 6 (8 times module C).

In an example, the acidic formulation comprises a silk polypeptide and formate, wherein the concentration of the silk polypeptide in the formulation is in the range of 0.25% by weight to 45% by weight, wherein the formulation has a pH which is equal or higher than the pI of the silk polypeptide, wherein the silk polypeptide is comprised in the formulation in an aggregated form and has a fibrillary structure, and wherein the silk polypeptide is selected from the group consisting of (C)ₘ, (C^{Cys})ₘ, (C^{Lys})ₘ, (C)ₘC^{Cys}, C^{Cys}(C)ₘ, C^{Lys}(C)ₘ, and (C)ₘC^{Lys}, wherein m is an integer of 2 to 96, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96. The silk polypeptide is exemplarily selected from the group consisting of C^{Lys}C₁₆, C^{Lys}C₃₂, C^{Lys}C₄₈, C₁₆C^{Lys}, C₃₂C^{Lys}, C₄₈C^{Lys}, C₁₆, C₃₂, C₄₈, C^{Cys}C₁₆, C^{Cys}C₃₂, C^{Cys}C₈, C₁₆C^{Cys}, C₃₂C^{Cys}, and C₄₈C^{Cys}.

In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

As to the specific ranges of the silk polypeptide concentration, it is referred to the above embodiments.

It is (additionally or alternatively) further preferred that the concentration of the formate in the acidic formulation is in the range of 10% by weight to 90% by weight. In particular, the concentration of the formate in this formulation is more preferably in the range of 10% by weight to 80% by weight, even more preferably in the range of 20% by weight to 75% by weight, still even more preferably in the range of 20% by weight to 70% by weight, and most preferably in the range of 25% by weight to 70% by weight. It is particularly preferred that the concentration of the silk polypeptide in this formulation is at least 0.5% by weight, 5% by weight, 10% by weight, or 20% by weight.

Thus, in one particularly preferred embodiment, the present invention relates to an acidic formulation comprising a silk polypeptide and formate, wherein the concentration of the silk polypeptide in the formulation is in the range of 0.25% by weight to 45% by weight, and wherein the concentration of the formate in the formulation is in the range of 10% by weight to 90% by weight.

Preferably, the concentration of the silk polypeptide in this formulation is in the range of 0.5% by weight to 40% by weight, more preferably in the range of 5% by weight to 30% by weight, even more preferably in the range of 5% by weight to 25% by weight, and most preferably in the range of 5% by weight to 20% by weight, and/or the concentration of the formate in this formulation is in the range of 10% by weight to 80% by weight, more preferably in the range of 20% by weight to 75% by weight, even more preferably in the range of 20% by weight to 70% by weight, and most preferably in the range of 25% by weight to 70% by weight.

Specifically, the concentration of the silk polypeptide in this formulation is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. More specifically, the concentration of the silk polypeptide in this formulation is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In one particularly more preferred embodiment, the present invention relates to an acidic formulation comprising a silk polypeptide and formate, wherein the concentration of the silk polypeptide in the formulation is in the range of 0.25% by weight to 45% by weight, wherein the concentration of the formate in the formulation is in the range of 10% by weight to 90% by weight, and wherein the silk polypeptide is comprised in the formulation in an aggregated form.

Preferably,
the concentration of the silk polypeptide in this formulation is in the range of 0.5% by weight to 40% by weight, more preferably in the range of 5% by weight to 30% by weight, even more preferably in the range of 5% by weight to 25% by weight, and most preferably in the range of 5% by weight to 20% by weight, and/or the concentration of the formate in this formulation is in the range of 10% by weight to 80% by weight, more preferably in the range of 20% by weight to 75% by weight, even more preferably in the range of 20% by weight to 70% by weight, and most preferably in the range of 25% by weight to 70% by weight.

Specifically, the concentration of the silk polypeptide in this formulation is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. More specifically, the concentration of the silk polypeptide in this formulation is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In one particularly even more preferred embodiment, the present invention relates to an acidic formulation comprising a silk polypeptide and formate, wherein the concentration of the silk polypeptide in the formulation is in the range of 0.25% by weight to 45% by weight, wherein the concentration of the formate in the formulation is in the range of 10% by weight to 90% by weight, and wherein the silk polypeptide is comprised in the formulation in an aggregated form and has a fibrillary structure.

Preferably,
the concentration of the silk polypeptide in this formulation is in the range of 0.5% by weight to 40% by weight, more preferably in the range of 5% by weight to 30% by weight, even more preferably in the range of 5% by weight to 25% by weight, and most preferably in the range of 5% by weight to 20% by weight, and/or the concentration of the formate in this formulation is in the range of 10% by weight to 80% by weight, more preferably in the range of 20% by weight to 75% by weight, even more preferably in the range of 20% by weight to 70% by weight, and most preferably in the range of 25% by weight to 70% by weight.

Specifically, the silk polypeptide in this formulation is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. More specifically, the concentration of the silk polypeptide in this formulation is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In one particularly still even more preferred embodiment, the present invention relates to an acidic formulation comprising a silk polypeptide and formate, wherein the concentration of the silk polypeptide in the formulation is in the range of 0.25% by weight to 45% by weight, wherein the concentration of the formate in the formulation is in the range of 10% by weight to 90% by weight, wherein the formulation has a pH which is equal or higher than the pI of the silk polypeptide, and wherein the silk polypeptide is comprised in the formulation in an aggregated form and has a fibrillary structure. In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

Preferably,
the concentration of the silk polypeptide in this formulation is in the range of 0.5% by weight to 40% by weight, more preferably in the range of 5% by weight to 30% by weight, even more preferably in the range of 5% by weight to 25% by weight, and most preferably in the range of 5% by weight to 20% by weight, and/or the concentration of the formate in this formulation is in the range of 10% by weight to 80% by weight, more preferably in the range of 20% by weight to 75% by weight, even more preferably in the range of 20% by weight to 70% by weight, and most preferably in the range of 25% by weight to 70% by weight.

Specifically, the concentration of the silk polypeptide in this formulation is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. Specifically, the concentration of the silk polypeptide in this formulation is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In an example, the acidic formulation comprises a silk polypeptide and formate, wherein the concentration of the silk polypeptide in the formulation is in the range of 0.25% by weight to 45% by weight, wherein the concentration of the formate in the formulation is in the range of 10% by weight to 90% by weight, wherein the formulation has a pH which is equal or higher than the pI of the silk polypeptide, wherein the silk polypeptide is comprised in the formulation in an aggregated form and has a fibrillary structure, and wherein the silk polypeptide is selected from the group consisting of (C)ₘ, (C^{Cys})ₘ, (C^{Lys})ₘ, (C)ₘC^{Cys}, C^{Cys}(C)ₘ, C^{Lys}(C)ₘ, and (C)ₘC^{Lys}, wherein m is an integer of 2 to 96, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96. The silk polypeptide is exemplarily selected from the group consisting of C^{Lys}C₁₆, C^{Lys}C₃₂, C^{Lys}C₄₈, C₁₆C^{Lys}, C₃₂C^{Lys}, C₄₈C^{Lys}, C₁₆, C₃₂, C₄₈, C^{Cys}C₁₆, C^{Cys}C₃₂, C^{Cys}C₈, C₁₆C^{Cys}, C₃₂C^{Cys}, and C₄₈C^{Cys}.

In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

As to the specific ranges of the silk polypeptide concentration, it is referred to the above embodiments.

The acidic formulation may be an aqueous acidic formulation, a buffered acidic formulation or a protein concentrate. The acidic formulation may have (additionally or alternatively) a gel-like structure. It is preferred that the gel-like structure is a hydrogel. The hydrogel may be composed of a buffered acidic formulation or protein concentrate.

Thus, in one particularly preferred embodiment, the present invention relates to a hydrogel comprising a silk polypeptide and formate, wherein the concentration of the silk polypeptide in the hydrogel is in the range of 0.25% by weight to 45% by weight.

Preferably, the concentration of the silk polypeptide in the hydrogel is in the range of 0.5% by weight to 40% by weight, more preferably in the range of 5% by weight to 30% by weight, even more preferably in the range of 5% by weight to 25% by weight, and most preferably in the range of 5% by weight to 20% by weight.

Specifically, the concentration of the silk polypeptide in the hydrogel is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. More specifically, the concentration of the silk polypeptide in the hydrogel is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In one particularly more preferred embodiment, the present invention relates to a hydrogel comprising a silk polypeptide and formate, wherein the concentration of the silk polypeptide in the hydrogel is in the range of 0.25% by weight to 45% by weight, and wherein the silk polypeptide is comprised in the hydrogel in an aggregated form.

Preferably, the concentration of the silk polypeptide in the hydrogel is in the range of 0.5% by weight to 40% by weight, more preferably in the range of 5% by weight to 30% by weight, even more preferably in the range of 5% by weight to 25% by weight, and most preferably in the range of 5% by weight to 20% by weight.

Specifically, the concentration of the silk polypeptide in the hydrogel is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. More specifically, the concentration of the silk polypeptide in the hydrogel is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In one particularly even more preferred embodiment, the present invention relates to a hydrogel comprising a silk polypeptide and formate, wherein the concentration of the silk polypeptide in the hydrogel is in the range of 0.25% by weight to 45% by weight, and wherein the silk polypeptide is comprised in the hydrogel in an aggregated form and has a fibrillary structure.

Preferably, the concentration of the silk polypeptide in the hydrogel is in the range of 0.5% by weight to 40% by weight, more preferably in the range of 5% by weight to 30% by weight, even more preferably in the range of 5% by weight to 25% by weight, and most preferably in the range of 5% by weight to 20% by weight.

Specifically, the concentration of the silk polypeptide in the hydrogel is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. More specifically, the concentration of the silk polypeptide in the hydrogel is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In one particularly still even more preferred embodiment, the present invention relates to a hydrogel comprising a silk polypeptide and formate, wherein the concentration of the silk polypeptide in the hydrogel is in the range of 0.25% by weight to 45% by weight, wherein the hydrogel has a pH which is equal or higher than the pI of the silk polypeptide, and wherein the silk polypeptide is comprised in the hydrogel in an aggregated form and has a fibrillary structure. In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

Preferably, the concentration of the silk polypeptide in the hydrogel is in the range of 0.5% by weight to 40% by weight, more preferably in the range of 5% by weight to 30% by weight, even more preferably in the range of 5% by weight to 25% by weight, and most preferably in the range of 5% by weight to 20% by weight.

Specifically, the concentration of the silk polypeptide in the hydrogel is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. More specifically, the concentration of the silk polypeptide in the hydrogel is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In one particularly most preferred embodiment, the present invention relates to a hydrogel comprising a silk polypeptide and formate, wherein the concentration of the silk polypeptide in the hydrogel is in the range of 0.25% by weight to 45% by weight, wherein the concentration of the formate in the hydrogel is in the range of 10% by weight to 90% by weight, wherein the hydrogel has a pH which is equal or higher than the pI of the silk polypeptide, and wherein the silk polypeptide is comprised in the hydrogel in an aggregated form and has a fibrillary structure. In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

Preferably,
the concentration of the silk polypeptide in the hydrogel is in the range of 0.5% by weight to 40% by weight, more preferably in the range of 5% by weight to 30% by weight, even more preferably in the range of 5% by weight to 25% by weight, and most preferably in the range of 5% by weight to 20% by weight, and/or
the concentration of the formate in the hydrogel is in the range of 10% by weight to 80% by weight, more preferably in the range of 20% by weight to 75% by weight, even more preferably in the range of 20% by weight to 70% by weight, and most preferably in the range of 25% by weight to 70% by weight.

Specifically, the concentration of the silk polypeptide in the hydrogel is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. More specifically, the concentration of the silk polypeptide in the hydrogel is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In an example, the hydrogel comprises a silk polypeptide and formate, wherein the concentration of the silk polypeptide in the hydrogel is in the range of 0.25% by weight to 45% by weight, wherein the concentration of the formate in the hydrogel is in the range of 10% by weight to 90% by weight, wherein the hydrogel has a pH which is equal or higher than the pI of the silk polypeptide, wherein the silk polypeptide is comprised in the hydrogel in an aggregated form and has a fibrillary structure, and wherein the silk polypeptide is selected from the group consisting of (C), (C^{Cys})ₘ, (C^{Lys})ₘ, (C)ₘC^{Cys}, C^{Cys}(C)ₘ, C^{Lys}(C)ₘ, and (C)ₘC^{Lys}, wherein m is an integer of 2 to 96, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96. The silk polypeptide is exemplarily selected from the group consisting of C^{Lys}C₁₆, C^{Lys}C₃₂, C^{Lys}C₄₈, C₁₆C^{Lys}, C₃₂C^{Lys}, C₄₈C^{Lys}, C₁₆, C₃₂, C₄₈, C^{Cys}C₁₆, C^{Cys}C₃₂, C^{Cys}C₈, C₁₆C^{Cys}, C₃₂C^{Cys}, and C₄₈C^{Cys}.

In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

As to the specific ranges of the silk polypeptide concentration, it is referred to the above embodiments.

It is (additionally or alternatively) still further preferred that the acidic formulation is a flowable acidic formulation.

Thus, in one particularly preferred embodiment, the present invention relates to a flowable acidic formulation (e.g. a hydrogel) comprising a silk polypeptide and formate, wherein the concentration of the silk polypeptide in the formulation is in the range of 0.25% by weight to 4% by weight.

Preferably, the concentration of the silk polypeptide in this formulation is in the range of 0.5% by weight to 4% by weight and more preferably in the range of 0.5% by weight to 3.5% by weight.

Specifically, the concentration of the silk polypeptide in this formulation is at least 0.25, 0.5, 0.6, 0.65, 0.7, 0.75, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.25, 3.3, 3.4, 3.5, 3.6, 3.7, 3.8, 3.9% by weight or 4% by weight. More specifically, the concentration of the silk polypeptide in this formulation is at least 0.25, 0.5, 0.6, 0.65, 0.7, 0.75, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.25, 3.3, 3.4, 3.5, 3.6, 3.7, 3.8, 3.9% by weight and not more than 4% by weight.

In one particularly more preferred embodiment, the present invention relates to a flowable acidic formulation (e.g. a hydrogel) comprising a silk polypeptide and formate, wherein the concentration of the silk polypeptide in the formulation is in the range of 0.25% by weight to 4% by weight, and wherein the silk polypeptide is comprised in the formulation in an aggregated form.

Preferably, the concentration of the silk polypeptide in this formulation is in the range of 0.5% by weight to 4% by weight and more preferably in the range of 0.5% by weight to 3.5% by weight.

Specifically, the concentration of the silk polypeptide in this formulation is at least 0.25, 0.5, 0.6, 0.65, 0.7, 0.75, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.25, 3.3, 3.4, 3.5, 3.6, 3.7, 3.8, 3.9% by weight or 4% by weight. More specifically, the concentration of the silk polypeptide in this formulation is at least 0.25, 0.5, 0.6, 0.65, 0.7, 0.75, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.25, 3.3, 3.4, 3.5, 3.6, 3.7, 3.8, 3.9% by weight and not more than 4% by weight.

In one particularly even more preferred embodiment, the present invention relates to a flowable acidic formulation (e.g. a hydrogel) comprising a silk polypeptide and formate, wherein the concentration of the silk polypeptide in the formulation is in the range of 0.25% by weight to 4% by weight, and wherein the silk polypeptide is comprised in the formulation in an aggregated form and has a fibrillary structure.

Preferably, the concentration of the silk polypeptide in this formulation is in the range of 0.5% by weight to 4% by weight and more preferably in the range of 0.5% by weight to 3.5% by weight.

Specifically, the concentration of the silk polypeptide in this formulation is at least 0.25, 0.5, 0.6, 0.65, 0.7, 0.75, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.25, 3.3, 3.4, 3.5, 3.6, 3.7, 3.8, 3.9% by weight or 4% by weight. More specifically, the concentration of the silk polypeptide in this formulation is at least 0.25, 0.5, 0.6, 0.65, 0.7, 0.75, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.25, 3.3, 3.4, 3.5, 3.6, 3.7, 3.8, 3.9% by weight and not more than 4% by weight.

In one particularly still even more preferred embodiment, the present invention relates to a flowable acidic formulation (e.g. a hydrogel) comprising a silk polypeptide and formate, wherein the concentration of the silk polypeptide in the formulation is in the range of 0.25% by weight to 4% by weight, wherein the formulation has a pH which is equal or higher than the pI of the silk polypeptide, and wherein the silk polypeptide is comprised in the formulation in an aggregated form and has a fibrillary structure. In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

Preferably, the concentration of the silk polypeptide in this formulation is in the range of 0.5% by weight to 4% by weight and more preferably in the range of 0.5% by weight to 3.5% by weight.

Specifically, the concentration of the silk polypeptide in this formulation is at least 0.25, 0.5, 0.6, 0.65, 0.7, 0.75, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.25, 3.3, 3.4, 3.5, 3.6, 3.7, 3.8, 3.9% by weight or 4% by weight. More specifically, the concentration of the silk polypeptide in this formulation is at least 0.25, 0.5, 0.6, 0.65, 0.7, 0.75, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.25, 3.3, 3.4, 3.5, 3.6, 3.7, 3.8, 3.9% by weight and not more than 4% by weight.

In one particularly most preferred embodiment, the present invention relates to a flowable acidic formulation (e.g. a hydrogel) comprising a silk polypeptide and formate, wherein the concentration of the silk polypeptide in the formulation is in the range of 0.25% by weight to 4% by weight, wherein the concentration of the formate in the hydrogel is in the range of 10% by weight to 90% by weight, wherein the hydrogel has a pH which is equal or higher than the pI of the silk polypeptide, and wherein the silk polypeptide is comprised in the formulation in an aggregated form and has a fibrillary structure. In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

Preferably,
the concentration of the formate in this formulation is in the range of 10% by weight to 80% by weight, more preferably in the range of 20% by weight to 75% by weight, even more preferably in the range of 20% by weight to 70% by weight, and most preferably in the range of 25% by weight to 70% by weight, and/or
the concentration of the silk polypeptide in this formulation is in the range of 0.5% by weight to 4% by weight and more preferably in the range of 0.5% by weight to 3.5% by weight.

Specifically, the concentration of the silk polypeptide in this formulation is at least 0.25, 0.5, 0.6, 0.65, 0.7, 0.75, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.25, 3.3, 3.4, 3.5, 3.6, 3.7, 3.8, 3.9% by weight or 4% by weight. More specifically, the concentration of the silk polypeptide in this formulation is at least 0.25, 0.5, 0.6, 0.65, 0.7, 0.75, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.25, 3.3, 3.4, 3.5, 3.6, 3.7, 3.8, 3.9% by weight and not more than 4% by weight.

In an example, the flowable acidic formulation comprises a silk polypeptide and formate, wherein the concentration of the silk polypeptide in the formulation is in the range of 0.25% by weight to 4% by weight, wherein the concentration of the formate in the formulation is in the range of 10% by weight to 90% by weight, wherein the formulation has a pH which is equal or higher than the pI of the silk polypeptide, wherein the silk polypeptide is comprised in the formulation in an aggregated form and has a fibrillary structure, and wherein the silk polypeptide is selected from the group consisting of (C)ₘ, (C^{Cys})ₘ, (C^{Lys})ₘ, (C)ₘC^{Cys}, C^{Cys}(C)ₘ, C^{Lys}(C)ₘ, and (C)ₘC^{Lys}, wherein m is an integer of 2 to 96, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96. The silk polypeptide is exemplarily selected from the group consisting of C^{Lys}C₁₆, C^{Lys}C₃₂, C^{Lys}C₄₈, C₁₆C^{Lys}, C₃₂C^{Lys}, C₄₈C^{Lys}, C₁₆, C₃₂, C₄₈, C^{Cys}C₁₆, C^{Cys}C₃₂, C^{Cys}C₈, C₁₆C^{Cys}, C₃₂C^{Cys}, and C₄₈C^{Cys}.

As to the specific ranges of the silk polypeptide concentration, it is referred to the above embodiments.

Alternatively, the acidic formulation is a non-flowable/solid acidic formulation.

Thus, in one particularly preferred embodiment, the present invention relates to a non-flowable/solid acidic formulation (e.g. a hydrogel) comprising a silk polypeptide and formate, wherein the concentration of the silk polypeptide in the formulation is in the range of >4% by weight to 45% by weight.

Preferably,
the concentration of the silk polypeptide in this formulation is in the range of 5% by weight to 40% by weight, more preferably in the range of 5% by weight to 30% by weight, even more preferably in the range of 5% by weight to 25% by weight, and most preferably in the range of 5% by weight to 20% by weight.

Specifically, the concentration of the silk polypeptide in this formulation is at least 5, 6, 7, 7.7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39% by weight or 40% by weight. More specifically, the concentration of the silk polypeptide in this formulation is at least 5, 6, 7, 7.7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In one particularly more preferred embodiment, the present invention relates to a non-flowable/solid acidic formulation (e.g. a hydrogel) comprising a silk polypeptide and formate, wherein the concentration of the silk polypeptide in the formulation is in the range of >4% by weight to 45% by weight, and wherein the silk polypeptide is comprised in the formulation in an aggregated form.

Preferably, the concentration of the silk polypeptide in this formulation is in the range of 5% by weight to 40% by weight, more preferably in the range of 5% by weight to 30% by weight, even more preferably in the range of 5% by weight to 25% by weight, and most preferably in the range of 5% by weight to 20% by weight.

Specifically, the concentration of the silk polypeptide in this formulation is at least 5, 6, 7, 7.7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39% by weight or 40% by weight. More specifically, the concentration of the silk polypeptide in this formulation is at least 5, 6, 7, 7.7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In one particularly even more preferred embodiment, the present invention relates to a non-flowable/solid acidic formulation (e.g. a hydrogel) comprising a silk polypeptide and formate, wherein the concentration of the silk polypeptide in the formulation is in the range of >4% by weight to 45% by weight, and wherein the silk polypeptide is comprised in the formulation in an aggregated form and has a fibrillary structure.

Preferably, the concentration of the silk polypeptide in this formulation is in the range of 5% by weight to 40% by weight, more preferably in the range of 5% by weight to 30% by weight, even more preferably in the range of 5% by weight to 25% by weight, and most preferably in the range of 5% by weight to 20% by weight.

Specifically, the concentration of the silk polypeptide in this formulation is at least 5, 6, 7, 7.7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39% by weight or 40% by weight. More specifically, the concentration of the silk polypeptide in this formulation is at least 5, 6, 7, 7.7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In one particularly still even more preferred embodiment, the present invention relates to a non-flowable/solid acidic formulation (e.g. a hydrogel) comprising a silk polypeptide and formate, wherein the concentration of the silk polypeptide in the formulation is in the range of >4% by weight to 45% by weight, wherein the formulation has a pH which is equal or higher than the pI of the silk polypeptide, and wherein the silk polypeptide is comprised in the formulation in an aggregated form and has a fibrillary structure. In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

Preferably, the concentration of the silk polypeptide in this formulation is in the range of 5% by weight to 40% by weight, more preferably in the range of 5% by weight to 30% by weight, even more preferably in the range of 5% by weight to 25% by weight, and most preferably in the range of 5% by weight to 20% by weight.

Specifically, the concentration of the silk polypeptide in this formulation is at least 5, 6, 7, 7.7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39% by weight or 40% by weight. More specifically, the concentration of the silk polypeptide in this formulation is at least 5, 6, 7, 7.7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In one particularly most more preferred embodiment, the present invention relates to a non-flowable/solid acidic formulation (e.g. a hydrogel) comprising a silk polypeptide and formate, wherein the concentration of the silk polypeptide in the formulation is in the range of >4% by weight to 45% by weight, wherein the concentration of the formate in the hydrogel is in the range of 10% by weight to 90% by weight, wherein the hydrogel has a pH which is equal or higher than the pI of the silk polypeptide, and wherein the silk polypeptide is comprised in the formulation in an aggregated form and has a fibrillary structure. In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

Preferably,
the concentration of the formate in this formulation is in the range of 10% by weight to 80% by weight, more preferably in the range of 20% by weight to 75% by weight, even more preferably in the range of 20% by weight to 70% by weight, and most preferably in the range of 25% by weight to 70% by weight, and/or
the concentration of the silk polypeptide in this formulation is in the range of 5% by weight to 40% by weight, more preferably in the range of 5% by weight to 30% by weight, even more preferably in the range of 5% by weight to 25% by weight, and most preferably in the range of 5% by weight to 20% by weight.

Specifically, the concentration of the silk polypeptide in this formulation is at least 5, 6, 7, 7.7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39% by weight or 40% by weight. More specifically, the concentration of the silk polypeptide in this formulation is at least 5, 6, 7, 7.7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In an example, the non-flowable/solid acidic formulation comprises a silk polypeptide and formate, wherein the concentration of the silk polypeptide in the formulation is in the range of >4% by weight to 45% by weight, wherein the concentration of the formate in the formulation is in the range of 10% by weight to 90% by weight, wherein the formulation has a pH which is equal or higher than the pI of the silk polypeptide, wherein the silk polypeptide is comprised in the formulation in an aggregated form and has a fibrillary structure, and wherein the silk polypeptide is selected from the group consisting of (C)ₘ, (C^{Cys})ₘ, (C^{Lys})ₘ, (C)ₘC^{Cys}, C^{Cys}(C)ₘ, C^{Lys}(C)ₘ, and (C)ₘC^{Lys}, wherein m is an integer of 2 to 96, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96. The silk polypeptide is exemplarily selected from the group consisting of C^{Lys}C₁₆, C^{Lys}C₃₂, C^{Lys}C₄₈, C₁₆C^{Lys}, C₃₂C^{Lys}, C₄₈C^{Lys}, C₁₆, C₃₂, C₄₈, C^{Cys}C₁₆, C^{Cys}C₃₂, C^{Cys}C₈, C₁₆C^{Cys}, C₃₂C^{Cys}, and C₄₈C^{Cys}.

As to the specific ranges of the silk polypeptide concentration, it is referred to the above embodiments.

The (flowable or non-flowable/solid) acidic formulation such as hydrogel preferably further comprises a compound. Said compound may be incorporated into the (flowable or non-flowable/solid) acidic formulation such as hydrogel or attached to/coated onto the acidic formulation such as hydrogel. The compound may be any compound having a purpose which may be useful in the present invention, e.g. a compound useful in the agrochemical industry, cleaning industry, homecare industry, detergent industry, cosmetic industry or food industry. The compound useful in the agrochemical industry may be selected from the group consisting of dyestuff, odoriferous substance, sunscreen, fertilizer, pesticide, hormone, growth factor, and nutrient. The compound useful in the food industry may be selected from the group consisting of a nutrient, vitamin, aroma, and preservative. The compound useful in the cleaning industry and homecare industry may be selected from the group consisting of dyestuff, antimicrobial substance, antiviral substance, detergent, surfactant, enzyme, fragrance, alcohol, greying inhibitor, dye transfer inhibitor, metal complex, nitrile, inorganic compound, bleaching agent, tenside, fatty acid, carbonic acid, silicate, carbonate, polymer, silicone, and brightening agent.

Accordingly, in one even more preferred embodiment, the present invention relates to an (a flowable or non-flowable/solid) acidic formulation comprising a silk polypeptide, a compound (useful in the agrochemical industry, cleaning industry, homecare industry, or food industry), and formate, wherein the concentration of the silk polypeptide in the formulation is in the range of 0.25% by weight to 45% by weight, wherein the formulation has a pH which is equal or higher than the pI of the silk polypeptide, and wherein the silk polypeptide is comprised in the formulation in an aggregated form and has a fibrillary structure. In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

Preferably, the concentration of the silk polypeptide in this formulation is in the range of 0.5% by weight to 40% by weight, more preferably in the range of 5% by weight to 30% by weight, even more preferably in the range of 5% by weight to 25% by weight, and most preferably in the range of 5% by weight to 20% by weight.

It is particularly preferred that the concentration of the silk polypeptide in this formulation is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. It is more particularly preferred that the concentration of the silk polypeptide in this formulation is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

As to the specific concentrations of the silk polypeptide in the (flowable or non-flowable/solid) acidic formulation, it is referred to the above embodiments.

More preferably, the silk polypeptide is selected from the group consisting of (C)ₘ, (C^{Cys})ₘ, (C^{Lys})ₘ, (C)ₘC^{Cys}, C^{Cys}(C)ₘ, C^{Lys}(C)ₘ, and (C)ₘC^{Lys}, wherein m is an integer of 2 to 96, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96. For example, the silk polypeptide is selected from the group consisting of C^{Lys}C₁₆, C^{Lys}C₃₂, C^{Lys}C₄₈, C₁₆C^{Lys}, C₃₂C^{Lys}, C₄₈C^{Lys}, C₁₆, C₃₂, C₄₈, C^{Cys}C₁₆, C^{Cys}C₃₂, C^{Cys}C₈, C₁₆C^{Cys}, C₃₂C^{Cys}, and C₄₈C^{Cys}.

In one most preferred embodiment, the present invention relates to an (a flowable or non-flowable/solid) acidic formulation comprising a silk polypeptide, a compound (useful in the agrochemical industry or food industry), and formate, wherein the concentration of the silk polypeptide in the formulation is in the range of 0.25% by weight to 45% by weight, wherein the concentration of the formate in the hydrogel is in the range of 10% by weight to 90% by weight, wherein the formulation has a pH which is equal or higher than the pI of the silk polypeptide, and wherein the silk polypeptide is comprised in the formulation in an aggregated form and has a fibrillary structure. In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

Preferably,
the concentration of the formate in this formulation is in the range of 10% by weight to 80% by weight, more preferably in the range of 20% by weight to 75% by weight, even more preferably in the range of 20% by weight to 70% by weight, and most preferably in the range of 25% by weight to 70% by weight, and/or
the concentration of the silk polypeptide in this formulation is in the range of 0.5% by weight to 40% by weight, more preferably in the range of 5% by weight to 30% by weight, even more preferably in the range of 5% by weight to 25% by weight, and most preferably in the range of 5% by weight to 20% by weight.

It is particularly preferred that the concentration of the silk polypeptide in this formulation is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. It is more particularly preferred that the concentration of the silk polypeptide in this formulation is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

As to the specific concentrations of the silk polypeptide in the (flowable or non-flowable/solid) acidic formulation, it is referred to the above embodiments.

More preferably, the silk polypeptide is selected from the group consisting of (C)ₘ, (C^{Cys})ₘ, (C^{Lys})ₘ, (C)ₘC^{Cys}, C^{Cys}(C)ₘ, C^{Lys}(C)ₘ, and (C)ₘC^{Lys}, wherein m is an integer of 2 to 96, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96. For example, the silk polypeptide is selected from the group consisting ofC^{Lys}C₁₆, C^{Lys}C₃₂, C^{Lys}C₄₈, C₁₆C^{Lys}, C₃₂C^{Lys}, C₄₈C^{Lys}, C₁₆, C₃₂, C₄₈, C^{Cys}C₁₆, C^{Cys}C₃₂, C^{Cys}C₈, C₁₆C^{Cys}, C₃₂C^{Cys}, and C₄₈C^{Cys}.

It is particularly preferred that the (flowable or non-flowable/solid) acidic formulation, e.g. hydrogel, of the first aspect is obtainable by the method of the second aspect. It is particularly more preferred that the (flowable or non-flowable/solid) acidic formulation, e.g. hydrogel, of the first aspect, is produced by the method of the second aspect.

The present inventors found that silk polypeptides can be solubilized in formic acid at high concentrations. Subsequently, an acidic formulation can be formed by setting a pH which is equal or higher than the pI of the silk polypeptide by titration. With suitable handling, a gel-like structure is produced within a short period of time. In this way various novel silk polypeptide formulations are produced. This behaviour is surprising, since in classical refolding processes, a titration to the pI of a polypeptide (e.g. neutralization) usually results in precipitation or crystallization of the polypeptide. A conformational change towards a gel-like structure is not yet known. Furthermore, in classical refolding processes that lead to a functional structure of the protein, the solvent usually has to be removed before a functional conformation can be achieved. This is not the case here.

Thus, in a second aspect, the present invention relates to a method for producing an acidic formulation comprising the steps of:
(i) providing an acidic solution comprising a silk polypeptide and formic acid, wherein the concentration of the silk polypeptide in the solution is in the range of 0.5% by weight to 45% by weight, and
(ii) forming an acidic formulation from the solution.

In particular, the acidic solution is an aqueous acidic solution. The aqueous acidic solution comprises, in addition to a silk polypeptide and formic acid, water.

The acidic solution comprising a silk polypeptide and formic acid provided in step (i) may be produced by adding formic acid to a silk polypeptide or by adding a silk polypeptide to formic acid. The silk polypeptide may be comprised in an aqueous solution or may be in the form of a powder or other dry state. The formic acid may be concentrated or may be comprised in an aqueous solution. Alternatively, both components (silk polypeptide and formic acid) are added together at the same time. Subsequently, the different components of the solution are mixed with each other. The acidic formulation which is formed in step (ii) comprises the silk polypeptide and formate. Formate is the salt of formic acid. It is produced by increasing the pH to neutralization.

Preferably, the concentration of the silk polypeptide in the acidic solution is in the range of 0.5% by weight to 40% by weight, more preferably in the range of 5% by weight to 30% by weight, even more preferably in the range of 5% by weight to 25% by weight, and most preferably in the range of 5% by weight to 20% by weight.

It is particularly preferred that the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. It is more particularly preferred that the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

It is preferred that the silk polypeptide is comprised in the formulation in an aggregated form and/or has a fibrillary structure. The silk polypeptide aggregation is the result of silk polypeptide self-assembly.

Accordingly, in one preferred embodiment, the present invention relates to a method for producing an acidic formulation comprising the steps of:
(i) providing an acidic solution comprising a silk polypeptide and formic acid, wherein the concentration of the silk polypeptide in the solution is in the range of 0.5% by weight to 45% by weight, and
(ii) forming an acidic formulation from the solution, wherein the silk polypeptide is comprised in the formulation in an aggregated form and/or has a fibrillary structure.

Preferably, the concentration of the silk polypeptide in the acidic solution is in the range of 0.5% by weight to 40% by weight, more preferably in the range of 5% by weight to 30% by weight, even more preferably in the range of 5% by weight to 25% by weight, and most preferably in the range of 5% by weight to 20% by weight.

It is particularly preferred that the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. It is more particularly preferred that the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

It is (alternatively or additionally) further preferred that the acidic formulation is formed by setting a pH which is equal or higher than the pI of the silk polypeptide. In case of the silk polypeptide C₁₆, as an example, this means the following: The silk polypeptide C₁₆ has a pI of 3.48. Thus, the acidic formulation comprising the silk polypeptide C₁₆ is formed by setting a pH which is equal or higher than 3.48. In particular, the pH is at least 0.1 pH units higher than the pI of the silk polypeptide. Preferably, the pH is at least 0.2, 0.3, 0.4, 0.5, 0.6, 0.7, 0.8, 0.9, 1, 2, 3, 4, 5, 6, 7, or 8 pH units higher than the pI of the silk polypeptide. In case of the silk polypeptide C₁₆, as an example, this means the following: The silk polypeptide C₁₆ has a pI of 3.48. Thus, the acidic formulation comprising the silk polypeptide C₁₆ is formed by setting a pH which is at least 3.58, at least 3.68, at least 3.78, at least 3.88, at least 3.98, at least 4.08, at least 4.18, at least 4.28, at least 4.38, at least 4.48, at least 5.48, at least 6.48, at least 7.48, at least 8.48, or at least 9.48. More preferably, the acidic formulation comprising the silk polypeptide C₁₆ is formed by setting a pH which is between 3.5 and 5. In one alternative, the acidic formulation comprising the silk polypeptide C₁₆ has a pH which is between 5 and 7. In one another alternative, the acidic formulation comprising the silk polypeptide C₁₆ has a pH which is between 7 and 10.

Accordingly, in one further preferred embodiment, the present invention relates to a method for producing an acidic formulation comprising the steps of:
(i) providing an acidic solution comprising a silk polypeptide and formic acid, wherein the concentration of the silk polypeptide in the solution is in the range of 0.5% by weight to 45% by weight, and
(ii) forming an acidic formulation from the solution by setting a pH which is equal or higher than the pI of the silk polypeptide, wherein the silk polypeptide is comprised in the formulation in an aggregated form and/or has a fibrillary structure.

The adaption of the pH may lead to neutralization. A formulation after neutralization may not be designated as acidic anymore. The acidic formulation after neutralization may simply be designated as formulation which comprises water, i.e. an aqueous formulation.

In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

Preferably, the concentration of the silk polypeptide in the acidic solution is in the range of 0.5% by weight to 40% by weight, more preferably in the range of 5% by weight to 30% by weight, even more preferably in the range of 5% by weight to 25% by weight, and most preferably in the range of 5% by weight to 20% by weight.

It is particularly preferred that the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. It is more particularly preferred that the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

The formation of the acidic formulation from the acidic solution may take place in a reaction container, reaction tank, or mould. For forming the acidic formulation, the acidic solution may also be cast on a flat surface, in a mould, or on a surface of a substrate.

It is (alternatively or additionally) still further preferred that the pH is set by adding a basic buffer or a base to the solution. The basic buffer may be selected from the group consisting of a buffer comprising sodium hydroxide (NaOH), potassium hydroxide (KOH). The base may be selected from the group consisting of sodium hydroxide (NaOH), potassium hydroxide (KOH).

Accordingly, in one more preferred embodiment, the present invention relates to a method for producing an acidic formulation comprising the steps of:
(i) providing an acidic solution comprising a silk polypeptide and formic acid, wherein the concentration of the silk polypeptide in the solution is in the range of 0.5% by weight to 45% by weight, and
(ii) forming an acidic formulation from the solution by setting a pH which is equal or higher than the pI of the silk polypeptide, wherein the pH is set by adding a basic buffer or a base to the solution, and wherein the silk polypeptide is comprised in the formulation in an aggregated form and/or has a fibrillary structure.

In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

Preferably, the concentration of the silk polypeptide in the acidic solution is in the range of 0.5% by weight to 40% by weight, more preferably in the range of 5% by weight to 30% by weight, even more preferably in the range of 5% by weight to 25% by weight, and most preferably in the range of 5% by weight to 20% by weight.

It is particularly preferred that the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. It is more particularly preferred that the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

It is (alternatively or additionally) also preferred that the silk polypeptide is a recombinant silk polypeptide. The (recombinant) silk polypeptide may be a spider silk polypeptide, e.g. a major ampullate silk polypeptide such as a dragline silk polypeptide, a minor ampullate silk polypeptide, or a flagelliform silk polypeptide of an orb-web spider. Particularly, the silk polypeptide is a spider silk polypeptide. More particularly, the spider silk polypeptide is a recombinant spider silk polypeptide.

It is particularly preferred that the (recombinant) silk polypeptide is a polypeptide with an amino acid sequence which comprises or consists of at least 50%, 60%, 65%, 70%, 75%, 80%, 85%, or 90% multiple copies of repetitive units. It is more preferred that the silk polypeptide is a polypeptide with an amino acid sequence which comprises or consists of at least 95% multiple copies of repetitive units. Said repetitive units may be identical or different.

It is particularly also preferred that the (recombinant) silk polypeptide consists of between 40 to 4000 amino acids. It is more preferred that the (recombinant) silk polypeptide consists of between 100 to 3500 amino acids or between 200 to 2500 amino acids. It is even more preferred that the (recombinant) silk polypeptide consists of between 250 to 2000 amino acids.

It is particularly further preferred that the (recombinant) silk polypeptide comprises at least two identical repetitive units. For example, the (recombinant) silk polypeptide may comprise between 2 to 100 repetitive units, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 repetitive units.

It is particularly more preferred that the repetitive units are independently selected from the group consisting of module C (SEQ ID NO: 1) or variants thereof, module C^{Cys} (SEQ ID NO: 2), and module C^{Lys} (SEQ ID NO: 7). Module C^{Cys} (SEQ ID NO: 2) is a variant of module C (SEQ ID NO: 1). Module C^{Lys} (SEQ ID NO: 7) is also a variant of module C (SEQ ID NO: 1). As to the different C module variants and fragments, it is referred to the first aspect of the present invention.

It is particularly even more preferred that the silk polypeptide is selected from the group consisting of (C)ₘ, (C^{Cys})ₘ, (C^{Lys})ₘ, (C)ₘC^{Cys}, C^{Cys}(C)ₘ, C^{Lys}(C)ₘ, and (C)ₘC^{Lys}, wherein m is an integer of 2 to 96, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96.

It is particularly most preferred that the silk polypeptide is selected from the group consisting of C^{Lys}C₁₆, C^{Lys}C₃₂, C^{Lys}C₄₈, C₁₆C^{Lys}, C₃₂C^{Lys}, C₄₈C^{Lys}, C₁₆, C₃₂, C₄₈, C^{Cys}C₁₆, C^{Cys}C₃₂, C^{Cys}C₈, C₁₆C^{Cys}, C₃₂C^{Cys}, and C₄₈C^{Cys}.

The silk polypeptide C₁₆ (16 times module C) has the amino acid sequence according to SEQ ID NO: 3, the silk polypeptide C₃₂ (32 times module C) has the amino acid sequence according to SEQ ID NO: 4, the silk polypeptide C₄₈ (48 times module C) has the amino acid sequence according to SEQ ID NO: 5, and the silk polypeptide C₈ has the amino acid sequence according to SEQ ID NO: 6 (8 times module C).

For example, the method for producing an acidic formulation comprises the steps of:
(i) providing an acidic solution comprising a silk polypeptide and formic acid, wherein the concentration of the silk polypeptide in the solution is in the range of 0.5% by weight to 45% by weight, and
(ii) forming an acidic formulation from the solution by setting a pH which is equal or higher than the pI of the silk polypeptide, wherein the pH is set by adding a basic buffer or a base to the solution, and wherein the silk polypeptide is comprised in the formulation in an aggregated form and/or has a fibrillary structure,
   wherein silk polypeptide is selected from the group consisting of (C)ₘ, (C^{Cys})ₘ, (C^{Lys})ₘ (C)ₘC^{Cys}, C^{Cys}(C)ₘ, C^{Lys}(C)ₘ, and (C)ₘC^{Lys}, wherein m is an integer of 2 to 96, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96. The silk polypeptide is exemplarily selected from the group consisting of C^{Lys}C₁₆, C^{Lys}C₃₂, C^{Lys}C₄₈, C₁₆C^{Lys}, C₃₂C^{Lys}, C₄₈C^{Lys}, C₁₆, C₃₂, C₄₈, C^{Cys}C₁₆, C^{Cys}C₃₂, C^{Cys}C₈, C₁₆C^{Cys}, C₃₂C^{Cys}, and C₄₈C^{Cys}.

In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

As to the specific ranges of the silk polypeptide concentration, it is referred to the above embodiments.

It is (alternatively or additionally) still also preferred that the concentration of the formic acid in the solution is in the range of 10% by weight to 90% by weight. In particular, the concentration of the formic acid in the solution is more preferably in the range of 10% by weight to 80% by weight, even more preferably in the range of 20% by weight to 75% by weight, still even more preferably in the range of 20% by weight to 70% by weight, and most preferably in the range of 25% by weight to 70% by weight. It is particularly preferred that the concentration of the silk polypeptide in this solution is at least 0.5% by weight, 5% by weight, 10% by weight, or 20% by weight.

Thus, in one particularly preferred embodiment, the present invention relates to a method for producing an acidic formulation comprising the steps of:
(i) providing an acidic solution comprising a silk polypeptide and formic acid, wherein the concentration of the silk polypeptide in the solution is in the range of 0.5% by weight to 45% by weight, and wherein the concentration of the formic acid in the solution is in the range of 10% by weight to 90% by weight, and
(ii) forming an acidic formulation from the solution.

Preferably, the concentration of the silk polypeptide in the acidic solution is in the range of 0.5% by weight to 40% by weight, more preferably in the range of 5% by weight to 30% by weight, even more preferably in the range of 5% by weight to 25% by weight, and most preferably in the range of 5% by weight to 20% by weight, and/or
the concentration of the formic acid in the acidic solution is in the range of 10% by weight to 80% by weight, more preferably in the range of 20% by weight to 75% by weight, even more preferably in the range of 20% by weight to 70% by weight, and most preferably in the range of 25% by weight to 70% by weight.

Specifically, the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. More specifically, the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In one particularly more preferred embodiment, the present invention relates to a method for producing an acidic formulation comprising the steps of:
(i) providing an acidic solution comprising a silk polypeptide and formic acid, wherein the concentration of the silk polypeptide in the solution is in the range of 0.5% by weight to 45% by weight, and wherein the concentration of the formic acid in the solution is in the range of 10% by weight to 90% by weight, and
(ii) forming an acidic formulation from the solution, wherein the silk polypeptide is comprised in the formulation in an aggregated form and/or has a fibrillary structure.

Preferably,
the concentration of the silk polypeptide in the acidic solution is in the range of 0.5% by weight to 40% by weight, more preferably in the range of 5% by weight to 30% by weight, even more preferably in the range of 5% by weight to 25% by weight, and most preferably in the range of 5% by weight to 20% by weight, and/or
the concentration of the formic acid in the acidic solution is in the range of 10% by weight to 80% by weight, more preferably in the range of 20% by weight to 75% by weight, even more preferably in the range of 20% by weight to 70% by weight, and most preferably in the range of 25% by weight to 70% by weight.

Specifically, the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. More specifically, the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In one particularly even more preferred embodiment, the present invention relates to a method for producing an acidic formulation comprising the steps of:
(i) providing an acidic solution comprising a silk polypeptide and formic acid, wherein the concentration of the silk polypeptide in the solution is in the range of 0.5% by weight to 45% by weight, and wherein the concentration of the formic acid in the solution is in the range of 10% by weight to 90% by weight, and
(ii) forming an acidic formulation from the solution by setting a pH which is equal or higher than the pI of the silk polypeptide, wherein the silk polypeptide is comprised in the formulation in an aggregated form and/or has a fibrillary structure.

In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

Preferably,
the concentration of the silk polypeptide in the acidic solution is in the range of 0.5% by weight to 40% by weight, more preferably in the range of 5% by weight to 30% by weight, even more preferably in the range of 5% by weight to 25% by weight, and most preferably in the range of 5% by weight to 20% by weight, and/or
the concentration of the formic acid in the acidic solution is in the range of 10% by weight to 80% by weight, more preferably in the range of 20% by weight to 75% by weight, even more preferably in the range of 20% by weight to 70% by weight, and most preferably in the range of 25% by weight to 70% by weight.

Specifically, the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. More specifically, the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In one particularly still even more preferred embodiment, the present invention relates to a method for producing an acidic formulation comprising the steps of:
(i) providing an acidic solution comprising a silk polypeptide and formic acid, wherein the concentration of the silk polypeptide in the solution is in the range of 0.5% by weight to 45% by weight, and wherein the concentration of the formic acid in the solution is in the range of 10% by weight to 90% by weight, and
(ii) forming an acidic formulation from the solution by setting a pH which is equal or higher than the pI of the silk polypeptide, wherein the pH is set by adding a buffer or a base to the solution, and wherein the silk polypeptide is comprised in the formulation in an aggregated form and/or has a fibrillary structure.

In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

Preferably,
the concentration of the silk polypeptide in the acidic solution is in the range of 0.5% by weight to 40% by weight, more preferably in the range of 5% by weight to 30% by weight, even more preferably in the range of 5% by weight to 25% by weight, and most preferably in the range of 5% by weight to 20% by weight, and/or
the concentration of the formic acid in the acidic solution is in the range of 10% by weight to 80% by weight, more preferably in the range of 20% by weight to 75% by weight, even more preferably in the range of 20% by weight to 70% by weight, and most preferably in the range of 25% by weight to 70% by weight.

Specifically, the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. More specifically, the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In an example, the method for producing an acidic formulation comprises the steps of:
(i) providing an acidic solution comprising a silk polypeptide and formic acid, wherein the concentration of the silk polypeptide in the solution is in the range of 0.5% by weight to 45% by weight, and wherein the concentration of the formic acid in the solution is in the range of 10% by weight to 90% by weight, and
(ii) forming an acidic formulation from the solution by setting a pH which is equal or higher than the pI of the silk polypeptide, wherein the pH is set by adding a buffer or a base to the solution, and wherein the silk polypeptide is comprised in the formulation in an aggregated form and/or has a fibrillary structure, wherein silk polypeptide is selected from the group consisting of (C)ₘ, (C^{Cys})ₘ, (C^{Lys})ₘ, (C)ₘC^{Cys}, C^{Cys}(C)ₘ, C^{Lys}(C)ₘ, and (C)ₘC^{Lys}, wherein m is an integer of 2 to 96, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96. The silk polypeptide is exemplarily selected from the group consisting of C^{Lys}C₁₆, C^{Lys}C₃₂, C^{Lys}C₄₈, C₁₆C^{Lys}, C₃₂C^{Lys}, C₄₈C^{Lys}, C₁₆, C₃₂, C₄₈, C^{Cys}C₁₆, C^{Cys}C₃₂, C^{Cys}C₈, C₁₆C^{Cys}, C₃₂C^{Cys}, and C₄₈C^{Cys}.

In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

As to the specific ranges of the silk polypeptide concentration, it is referred to the above embodiments.

The strong denaturing effect of formic acid additionally enables the dissolution and/or disruption of components of host cells. Thus, for applications with low purity requirements, surprisingly not only the purified protein powder can be used for the production of silk formulations, but also all intermediate stages from the fermentation broth to the polished powder in both dried and concentrated liquid form. Thus, under certain circumstances, most of or the complete costs for purification can be avoided. Accordingly, the acidic solution may be selected from the group consisting of an aqueous acidic solution, a buffered acidic solution, a fermentation broth, a protein concentrate, washed cells, a cell lysate, and an inclusion bodies suspension, or combinations thereof.

The present inventors surprisingly found that the formation of a gel-like structure and, thus, a functional structure of structural polypeptides is at all possible at high concentrations of the strong solvent formic acid (e.g. at >50%). Accordingly, the acidic solution may have (additionally or alternatively) a gel-like structure. It is preferred that the gel-like structure is a hydrogel. The hydrogel may be composed of a buffered acidic formulation or protein concentrate.

Thus, in one particularly preferred embodiment, the present invention relates to a method for producing a hydrogel comprising the steps of:
(i) providing an acidic solution comprising a silk polypeptide and formic acid, wherein the concentration of the silk polypeptide in the solution is in the range of 0.5% by weight to 45% by weight, and
(ii) forming a hydrogel from the solution.

Preferably, the concentration of the silk polypeptide in the acidic solution is in the range of 0.5% by weight to 40% by weight, more preferably in the range of 5% by weight to 30% by weight, even more preferably in the range of 5% by weight to 25% by weight, and most preferably in the range of 5% by weight to 20% by weight.

Specifically, the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. More specifically, the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In one particularly more preferred embodiment, the present invention relates to a method for producing a hydrogel comprising the steps of:
(i) providing an acidic solution comprising a silk polypeptide and formic acid, wherein the concentration of the silk polypeptide in the solution is in the range of 0.5% by weight to 45% by weight, and
(ii) forming a hydrogel from the solution, wherein the silk polypeptide is comprised in the hydrogel in an aggregated form and/or has a fibrillary structure.

Preferably, the concentration of the silk polypeptide in the acidic solution is in the range of 0.5% by weight to 40% by weight, more preferably in the range of 5% by weight to 30% by weight, even more preferably in the range of 5% by weight to 25% by weight, and most preferably in the range of 5% by weight to 20% by weight.

Specifically, the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. More specifically, the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In one particularly even more preferred embodiment, the present invention relates to a method for producing a hydrogel comprising the steps of:
(i) providing an acidic solution comprising a silk polypeptide and formic acid, wherein the concentration of the silk polypeptide in the solution is in the range of 0.5% by weight to 45% by weight, and
(ii) forming a hydrogel from the solution by setting a pH which is equal or higher than the pI of the silk polypeptide, wherein the silk polypeptide is comprised in the hydrogel in an aggregated form and/or has a fibrillary structure.

In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

Preferably, the concentration of the silk polypeptide in the acidic solution is in the range of 0.5% by weight to 40% by weight, more preferably in the range of 5% by weight to 30% by weight, even more preferably in the range of 5% by weight to 25% by weight, and most preferably in the range of 5% by weight to 20% by weight.

Specifically, the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. More specifically, the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In one particularly still even more preferred embodiment, the present invention relates to a method for producing a hydrogel comprising the steps of:
(i) providing an acidic solution comprising a silk polypeptide and formic acid, wherein the concentration of the silk polypeptide in the solution is in the range of 0.5% by weight to 45% by weight, and
(ii) forming a hydrogel from the solution by setting a pH which is equal or higher than the pI of the silk polypeptide, wherein the pH is set by adding a basic buffer or a base to the solution, and wherein the silk polypeptide is comprised in the hydrogel in an aggregated form and/or has a fibrillary structure.

In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

Preferably, the concentration of the silk polypeptide in the acidic solution is in the range of 0.5% by weight to 40% by weight, more preferably in the range of 5% by weight to 30% by weight, even more preferably in the range of 5% by weight to 25% by weight, and most preferably in the range of 5% by weight to 20% by weight.

Specifically, the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. More specifically, the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In one particularly most preferred embodiment, the present invention relates to a method for producing a hydrogel comprising the steps of:
(i) providing an acidic solution comprising a silk polypeptide and formic acid, wherein the concentration of the silk polypeptide in the solution is in the range of 0.5% by weight to 45% by weight, and wherein the concentration of the formic acid in the solution is in the range of 10% by weight to 90% by weight, and
(ii) forming a hydrogel from the solution by setting a pH which is equal or higher than the pI of the silk polypeptide, wherein the pH is set by adding a basic buffer or a base to the solution, and wherein the silk polypeptide is comprised in the hydrogel in an aggregated form and/or has a fibrillary structure.

In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

Preferably,
the concentration of the silk polypeptide in the acidic solution is in the range of 0.5% by weight to 40% by weight, more preferably in the range of 5% by weight to 30% by weight, even more preferably in the range of 5% by weight to 25% by weight, and most preferably in the range of 5% by weight to 20% by weight, and/or
the concentration of the formic acid in the acidic solution is in the range of 10% by weight to 80% by weight, more preferably in the range of 20% by weight to 75% by weight, even more preferably in the range of 20% by weight to 70% by weight, and most preferably in the range of 25% by weight to 70% by weight.

Specifically, the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. More specifically, the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In an example, the method for producing a hydrogel comprises the steps of:
(i) providing an acidic solution comprising a silk polypeptide and formic acid, wherein the concentration of the silk polypeptide in the solution is in the range of 0.5% by weight to 45% by weight, and wherein the concentration of the formic acid in the solution is in the range of 10% by weight to 90% by weight, and
(ii) forming a hydrogel from the solution by setting a pH which is equal or higher than the pI of the silk polypeptide, wherein the pH is set by adding a basic buffer or a base to the solution, and wherein the silk polypeptide is comprised in the hydrogel in an aggregated form and/or has a fibrillary structure, wherein silk polypeptide is selected from the group consisting of (C)ₘ, (C^{Cys})ₘ, (C^{Lys})ₘ, (C)ₘC^{Cys}, C^{Cys}(C)ₘ, C^{Lys}(C)ₘ, and (C)ₘC^{Lys}, wherein m is an integer of 2 to 96, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96. The silk polypeptide is exemplarily selected from the group consisting of C^{Lys}C₁₆, C^{Lys}C₃₂, C^{Lys}C₄₈, C₁₆C^{Lys}, C₃₂C^{Lys}, C₄₈C^{Lys}, C₁₆, C₃₂, C₄₈, C^{Cys}C₁₆, C^{Cys}C₃₂, C^{Cys}C₈, C₁₆C^{Cys}, C₃₂C^{Cys}, and C₄₈C^{Cys}.

In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

As to the specific ranges of the silk polypeptide concentration, it is referred to the above embodiments.

It is (additionally or alternatively) still further preferred that the acidic formulation which is formed in step (ii) is a flowable acidic formulation.

Thus, in one particularly preferred embodiment, the present invention relates to a method for producing a flowable acidic formulation (e.g. a hydrogel) comprising the steps of:
(i) providing an acidic solution comprising a silk polypeptide and formic acid, wherein the concentration of the silk polypeptide in the solution is in the range of 0.25% by weight to 4% by weight, and
(ii) forming a flowable acidic formulation (e.g. a hydrogel) from the solution.

Preferably, the concentration of the silk polypeptide in the acidic solution is in the range of 0.5% by weight to 4% by weight and more preferably in the range of 0.5% by weight to 3.5% by weight.

Specifically, the concentration of the silk polypeptide in in the acidic solution is at least 0.25, 0.5, 0.6, 0.65, 0.7, 0.75, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.25, 3.3, 3.4, 3.5, 3.6, 3.7, 3.8, 3.9% by weight or 4% by weight. More specifically, the concentration of the silk polypeptide in in the acidic solution is at least 0.25, 0.5, 0.6, 0.65, 0.7, 0.75, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.25, 3.3, 3.4, 3.5, 3.6, 3.7, 3.8, 3.9% by weight and not more than 4% by weight.

In one particularly more preferred embodiment, the present invention relates to a method for producing a flowable acidic formulation (e.g. a hydrogel) comprising the steps of:
(i) providing an acidic solution comprising a silk polypeptide and formic acid, wherein the concentration of the silk polypeptide in the solution is in the range of 0.25% by weight to 4% by weight, and
(ii) forming a flowable acidic formulation (e.g. a hydrogel) from the solution, wherein the silk polypeptide is comprised in the formulation in an aggregated form and/or has a fibrillary structure.

Preferably, the concentration of the silk polypeptide in the acidic solution is in the range of 0.5% by weight to 4% by weight and more preferably in the range of 0.5% by weight to 3.5% by weight.

Specifically, the concentration of the silk polypeptide in in the acidic solution is at least 0.25, 0.5, 0.6, 0.65, 0.7, 0.75, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.25, 3.3, 3.4, 3.5, 3.6, 3.7, 3.8, 3.9% by weight or 4% by weight. More specifically, the concentration of the silk polypeptide in in the acidic solution is at least 0.25, 0.5, 0.6, 0.65, 0.7, 0.75, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.25, 3.3, 3.4, 3.5, 3.6, 3.7, 3.8, 3.9% by weight and not more than 4% by weight.

In one particularly even more preferred embodiment, the present invention relates to a method for producing a flowable acidic formulation (e.g. a hydrogel) comprising the steps of:
(i) providing an acidic solution comprising a silk polypeptide and formic acid, wherein the concentration of the silk polypeptide in the solution is in the range of 0.25% by weight to 4% by weight, and
(ii) forming a hydrogel from the solution by setting a pH which is equal or higher than the pI of the silk polypeptide, wherein the silk polypeptide is comprised in the formulation in an aggregated form and/or has a fibrillary structure.

In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

Preferably, the concentration of the silk polypeptide in the acidic solution is in the range of 0.5% by weight to 4% by weight and more preferably in the range of 0.5% by weight to 3.5% by weight.

Specifically, the concentration of the silk polypeptide in in the acidic solution is at least 0.25, 0.5, 0.6, 0.65, 0.7, 0.75, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.25, 3.3, 3.4, 3.5, 3.6, 3.7, 3.8, 3.9% by weight or 4% by weight. More specifically, the concentration of the silk polypeptide in in the acidic solution is at least 0.25, 0.5, 0.6, 0.65, 0.7, 0.75, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.25, 3.3, 3.4, 3.5, 3.6, 3.7, 3.8, 3.9% by weight and not more than 4% by weight.

In one particularly still even more preferred embodiment, the present invention relates to a method for producing a flowable acidic formulation (e.g. a hydrogel) comprising the steps of:
(i) providing an acidic solution comprising a silk polypeptide and formic acid, wherein the concentration of the silk polypeptide in the solution is in the range of 0.25% by weight to 4% by weight, and
(ii) forming a flowable acidic formulation (e.g. a hydrogel) from the solution by setting a pH which is equal or higher than the pI of the silk polypeptide, wherein the pH is set by adding a basic buffer or a base to the solution, and wherein the silk polypeptide is comprised in the formulation in an aggregated form and/or has a fibrillary structure.

In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

Preferably, the concentration of the silk polypeptide in the acidic solution is in the range of 0.5% by weight to 4% by weight and more preferably in the range of 0.5% by weight to 3.5% by weight.

Specifically, the concentration of the silk polypeptide in in the acidic solution is at least 0.25, 0.5, 0.6, 0.65, 0.7, 0.75, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.25, 3.3, 3.4, 3.5, 3.6, 3.7, 3.8, 3.9% by weight or 4% by weight. More specifically, the concentration of the silk polypeptide in in the acidic solution is at least 0.25, 0.5, 0.6, 0.65, 0.7, 0.75, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.25, 3.3, 3.4, 3.5, 3.6, 3.7, 3.8, 3.9% by weight and not more than 4% by weight.

In one particularly most preferred embodiment, the present invention relates to a method for producing a flowable acidic formulation (e.g. a hydrogel) comprising the steps of:
(i) providing an acidic solution comprising a silk polypeptide and formic acid, wherein the concentration of the silk polypeptide in the solution is in the range of 0.25% by weight to 4% by weight, and wherein the concentration of the formic acid in the solution is in the range of 10% by weight to 90% by weight, and
(ii) forming a flowable acidic formulation (e.g. a hydrogel) from the solution by setting a pH which is equal or higher than the pI of the silk polypeptide, wherein the pH is set by adding a basic buffer or a base to the solution, and wherein the silk polypeptide is comprised in the formulation in an aggregated form and/or has a fibrillary structure.

In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

Preferably,
the concentration of the silk polypeptide in the acidic solution is in the range of 0.5% by weight to 4% by weight and more preferably in the range of 0.5% by weight to 3.5% by weight, and/or
the concentration of the formic acid in the acidic solution is in the range of 10% by weight to 80% by weight, more preferably in the range of 20% by weight to 75% by weight, even more preferably in the range of 20% by weight to 70% by weight, and most preferably in the range of 25% by weight to 70% by weight.

Specifically, the concentration of the silk polypeptide in in the acidic solution is at least 0.25, 0.5, 0.6, 0.65, 0.7, 0.75, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.25, 3.3, 3.4, 3.5, 3.6, 3.7, 3.8, 3.9% by weight or 4% by weight. More specifically, the concentration of the silk polypeptide in in the acidic solution is at least 0.25, 0.5, 0.6, 0.65, 0.7, 0.75, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.25, 3.3, 3.4, 3.5, 3.6, 3.7, 3.8, 3.9% by weight and not more than 4% by weight.

In an example, the method for producing a flowable acidic formulation (e.g. a hydrogel) comprises the steps of:
(i) providing an acidic solution comprising a silk polypeptide and formic acid, wherein the concentration of the silk polypeptide in the solution is in the range of 0.25% by weight to 4% by weight, and wherein the concentration of the formic acid in the solution is in the range of 10% by weight to 90% by weight, and
(ii) forming a flowable acidic formulation (e.g. a hydrogel) from the solution by setting a pH which is equal or higher than the pI of the silk polypeptide, wherein the pH is set by adding a basic buffer or a base to the solution, and wherein the silk polypeptide is comprised in the hydrogel in an aggregated form and/or has a fibrillary structure, wherein silk polypeptide is selected from the group consisting of (C)ₘ, (C^{Cys})ₘ, (C^{Lys})ₘ, (C)ₘC^{Cys}, C^{Cys}(C)ₘ, C^{Lys}(C)ₘ, and (C)ₘC^{Lys}, wherein m is an integer of 2 to 96, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96. The silk polypeptide is exemplarily selected from the group consisting of C^{Lys}C₁₆, C^{Lys}C₃₂, C^{Lys}C₄₈, C₁₆C^{Lys}, C₃₂C^{Lys}, C₄₈C^{Lys}, C₁₆, C₃₂, C₄₈, C^{Cys}C₁₆, C^{Cys}C₃₂, C^{Cys}C₈, C₁₆C^{Cys}, C₃₂C^{Cys}, and C₄₈C^{Cys}.

In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

As to the specific ranges of the silk polypeptide concentration, it is referred to the above embodiments.

Alternatively, the acidic formulation which is formed in step (ii) is a non-flowable/solid acidic formulation.

Thus, in one particularly preferred embodiment, the present invention relates to a method for producing a non-flowable/solid acidic formulation (e.g. a hydrogel) comprising the steps of:
(i) providing an acidic solution comprising a silk polypeptide and formic acid, wherein the concentration of the silk polypeptide in the solution is in the range of >4% by weight to 45% by weight, and
(ii) forming a non-flowable/solid acidic formulation (e.g. a hydrogel) from the solution.

Preferably, the concentration of the silk polypeptide in the acidic solution is in the range of 5% by weight to 40% by weight, more preferably in the range of 5% by weight to 30% by weight, even more preferably in the range of 5% by weight to 25% by weight, and most preferably in the range of 5% by weight to 20% by weight.

Specifically, the concentration of the silk polypeptide in the acidic solution is at least 5, 6, 7, 7.7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39% by weight or 40% by weight. More specifically, the concentration of the silk polypeptide in the acidic solution is at least 5, 6, 7, 7.7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In one particularly more preferred embodiment, the present invention relates to a method for producing a non-flowable/solid acidic formulation (e.g. a hydrogel) comprising the steps of:
(i) providing an acidic solution comprising a silk polypeptide and formic acid, wherein the concentration of the silk polypeptide in the solution is in the range of >4% by weight to 45% by weight, and
(ii) forming a non-flowable/solid acidic formulation (e.g. a hydrogel) from the solution, wherein the silk polypeptide is comprised in the formulation in an aggregated form and/or has a fibrillary structure.

Preferably, the concentration of the silk polypeptide in the acidic solution is in the range of 5% by weight to 40% by weight, more preferably in the range of 5% by weight to 30% by weight, even more preferably in the range of 5% by weight to 25% by weight, and most preferably in the range of 5% by weight to 20% by weight.

Specifically, the concentration of the silk polypeptide in the acidic solution is at least 5, 6, 7, 7.7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39% by weight or 40% by weight. More specifically, the concentration of the silk polypeptide in the acidic solution is at least 5, 6, 7, 7.7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In one particularly even more preferred embodiment, the present invention relates to a method for producing a non-flowable/solid acidic formulation (e.g. a hydrogel) comprising the steps of:
(i) providing an acidic solution comprising a silk polypeptide and formic acid, wherein the concentration of the silk polypeptide in the solution is in the range of >4% by weight to 45% by weight, and
(ii) forming a non-flowable/solid from the solution by setting a pH which is equal or higher than the pI of the silk polypeptide, wherein the silk polypeptide is comprised in the formulation in an aggregated form and/or has a fibrillary structure.

In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

Preferably, the concentration of the silk polypeptide in the acidic solution is in the range of 5% by weight to 40% by weight, more preferably in the range of 5% by weight to 30% by weight, even more preferably in the range of 5% by weight to 25% by weight, and most preferably in the range of 5% by weight to 20% by weight.

Specifically, the concentration of the silk polypeptide in the acidic solution is at least 5, 6, 7, 7.7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39% by weight or 40% by weight. More specifically, the concentration of the silk polypeptide in the acidic solution is at least 5, 6, 7, 7.7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In one particularly still even more preferred embodiment, the present invention relates to a method for producing a non-flowable/solid acidic formulation (e.g. a hydrogel) comprising the steps of:
(i) providing an acidic solution comprising a silk polypeptide and formic acid, wherein the concentration of the silk polypeptide in the solution is in the range of >4% by weight to 45% by weight, and
(ii) forming a non-flowable/solid acidic formulation (e.g. a hydrogel) from the solution by setting a pH which is equal or higher than the pI of the silk polypeptide, wherein the pH is set by adding a basic buffer or a base to the solution, and wherein the silk polypeptide is comprised in the formulation in an aggregated form and/or has a fibrillary structure.

In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

Preferably, the concentration of the silk polypeptide in the acidic solution is in the range of 5% by weight to 40% by weight, more preferably in the range of 5% by weight to 30% by weight, even more preferably in the range of 5% by weight to 25% by weight, and most preferably in the range of 5% by weight to 20% by weight.

Specifically, the concentration of the silk polypeptide in the acidic solution is at least 5, 6, 7, 7.7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39% by weight or 40% by weight. More specifically, the concentration of the silk polypeptide in the acidic solution is at least 5, 6, 7, 7.7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In one particularly most preferred embodiment, the present invention relates to a method for producing a non-flowable/solid acidic formulation (e.g. a hydrogel) comprising the steps of:
(i) providing an acidic solution comprising a silk polypeptide and formic acid, wherein the concentration of the silk polypeptide in the solution is in the range of >4% by weight to 45% by weight, and wherein the concentration of the formic acid in the solution is in the range of 10% by weight to 90% by weight, and
(ii) forming a non-flowable/solid acidic formulation (e.g. a hydrogel) from the solution by setting a pH which is equal or higher than the pI of the silk polypeptide, wherein the pH is set by adding a basic buffer or a base to the solution, and wherein the silk polypeptide is comprised in the formulation in an aggregated form and/or has a fibrillary structure.

In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

Preferably,
the concentration of the silk polypeptide in the acidic solution is in the range of 5% by weight to 40% by weight, more preferably in the range of 5% by weight to 30% by weight, even more preferably in the range of 5% by weight to 25% by weight, and most preferably in the range of 5% by weight to 20% by weight, and/or
the concentration of the formic acid in the acidic solution is in the range of 10% by weight to 80% by weight, more preferably in the range of 20% by weight to 75% by weight, even more preferably in the range of 20% by weight to 70% by weight, and most preferably in the range of 25% by weight to 70% by weight.

Specifically, the concentration of the silk polypeptide in the acidic solution is at least 5, 6, 7, 7.7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39% by weight or 40% by weight. More specifically, the concentration of the silk polypeptide in the acidic solution is at least 5, 6, 7, 7.7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In an example, the method for producing a non-flowable/solid acidic formulation (e.g. a hydrogel) comprises the steps of:
(i) providing an acidic solution comprising a silk polypeptide and formic acid, wherein the concentration of the silk polypeptide in the solution is in the range of >4% by weight to 45% by weight, and wherein the concentration of the formic acid in the solution is in the range of 10% by weight to 90% by weight, and
(ii) forming a non-flowable/solid acidic formulation (e.g. a hydrogel) from the solution by setting a pH which is equal or higher than the pI of the silk polypeptide, wherein the pH is set by adding a basic buffer or a base to the solution, and wherein the silk polypeptide is comprised in the hydrogel in an aggregated form and/or has a fibrillary structure, wherein silk polypeptide is selected from the group consisting of (C)ₘ, (C^{Cys})ₘ, (C^{Lys})ₘ, (C)ₘC^{Cys}, C^{Cys}(C)ₘ, C^{Lys}(C)ₘ, and (C)ₘC^{Lys}, wherein m is an integer of 2 to 96, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96. The silk polypeptide is exemplarily selected from the group consisting of C^{Lys}C₁₆, C^{Lys}C₃₂, C^{Lys}C₄₈, C₁₆C^{Lys}, C₃₂C^{Lys}, C₄₈C^{Lys}, C₁₆, C₃₂, C₄₈, C^{Cys}C₁₆, C^{Cys}C₃₂, C^{Cys}C₈, C₁₆C^{Cys}, C₃₂C^{Cys}, and C₄₈C^{Cys}.

In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

As to the specific ranges of the silk polypeptide concentration, it is referred to the above embodiments.

The (flowable or non-flowable/solid) acidic formulation, e.g. hydrogel, preferably further comprises a compound. The compound may be any compound having a purpose which may be useful in the present invention, e.g. a compound useful in the agrochemical industry, cleaning industry, home care industry, detergent industry, cosmetic industry, or food industry. The compound useful in the agrochemical industry may be selected from the group consisting of dyestuff, odoriferous substance, sunscreen, fertilizer, pesticide, hormone, growth factor, insecticide, pesticide, herbicide, fungizide, microorganisms, and nutrient. The compound useful in the food industry may be selected from the group consisting of a nutrient, vitamin, aroma, preservative and dye. The compound useful in the cleaning industry and homecare industry may be selected from the group consisting of dyestuff, antimicrobial substance, antiviral substance, detergent, surfactant, enzyme, fragrance, alcohol, greying inhibitor, dye transfer inhibitor, metal complex, nitrile, inorganic compound, bleaching agent, tenside, fatty acid, carbonic acid, silicate, carbonate, polymer, silicone, brightening agent.

The acidic solution can be contacted with the compound prior to forming the (flowable or non-flowable/solid) acidic formulation, e.g. hydrogel. For example, the compound is added to the acidic solution comprising a silk polypeptide and formic acid provided in step (i). In an alternative, the compound is added to the acidic solution comprising a silk polypeptide and afterwards, after a mixing step, formic acid is added. In another alternative, the compound is added to the acidic solution comprising formic acid and afterwards, after a mixing step, the silk polypeptide is added.

Thus, in one even more preferred embodiment, the present invention relates to a method for producing an (a flowable or non-flowable/solid) acidic formulation comprising the steps of:
(i) providing an acidic solution comprising a silk polypeptide and formic acid, wherein the concentration of the silk polypeptide in the solution is in the range of 0.5% by weight to 45% by weight,
(ii) adding a compound (useful in the agrochemical industry, cleaning industry, home care industry, detergent industry, cosmetic industry, or food industry) to the acidic solution comprising a silk polypeptide and formic acid, and
(iii) forming an (a flowable or non-flowable/solid) acidic formulation from the solution comprising a silk polypeptide, formic acid, and a compound, wherein the silk polypeptide is preferably comprised in the formulation in an aggregated form and/or has a fibrillary structure.

Preferably, the concentration of the silk polypeptide in the acidic solution is in the range of 0.5% by weight to 40% by weight, more preferably in the range of 5% by weight to 30% by weight, even more preferably in the range of 5% by weight to 25% by weight, and most preferably in the range of 5% by weight to 20% by weight.

It is particularly preferred that the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. It is more particularly preferred that the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

Alternatively, the compound may be loaded into, coated onto, or attached to the (flowable or non-flowable/solid) acidic formulation after it is formed. For loading, coating, or attaching after formation, the compound is applied to the (flowable or non-flowable/solid) acidic formulation after it is formed.

Thus, in one alternative even more preferred embodiment, the present invention relates to a method for producing an (a flowable or non-flowable/solid) acidic formulation comprising the steps of:
(i) providing an acidic solution comprising a silk polypeptide and formic acid, wherein the concentration of the silk polypeptide in the solution is in the range of 0.5% by weight to 45% by weight,
(ii) forming an (a flowable or non-flowable/solid) acidic formulation from the solution, wherein the silk polypeptide is preferably comprised in the formulation in an aggregated form and/or has a fibrillary structure, and
(iii) adding a compound (useful in the agrochemical industry, cleaning industry, home care industry, detergent industry, cosmetic industry, or food industry) to the (flowable or non-flowable/solid) acidic formulation.

Preferably, the concentration of the silk polypeptide in the acidic solution is in the range of 0.5% by weight to 40% by weight, more preferably in the range of 5% by weight to 30% by weight, even more preferably in the range of 5% by weight to 25% by weight, and most preferably in the range of 5% by weight to 20% by weight.

It is particularly preferred that the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight.

It is more particularly preferred that the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In one still even more preferred embodiment, the present invention relates to a method for producing an (a flowable or non-flowable/solid) acidic formulation comprising the steps of:
(i) providing an acidic solution comprising a silk polypeptide and formic acid, wherein the concentration of the silk polypeptide in the solution is in the range of 0.5% by weight to 45% by weight, and wherein the concentration of the formic acid in the solution is in the range of 10% by weight to 90% by weight,
(ii) adding a compound (useful in the agrochemical industry, cleaning industry, home care industry, detergent industry, cosmetic industry, or food industry) to the acidic solution comprising a silk polypeptide and formic acid, and
(ii) forming an (a flowable or non-flowable/solid) acidic formulation from the solution comprising a silk polypeptide, formic acid, and a compound preferably by setting a pH which is equal or higher than the pI of the silk polypeptide, wherein the pH is set by adding a basic buffer or a base to said solution, and wherein the silk polypeptide is comprised in the (flowable acidic or non-flowable/solid) formulation in an aggregated form and/or has a fibrillary structure.

In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

Preferably,
the concentration of the formic acid in the acidic solution is in the range of 10% by weight to 80% by weight, more preferably in the range of 20% by weight to 75% by weight, even more preferably in the range of 20% by weight to 70% by weight, and most preferably in the range of 25% by weight to 70% by weight, and/or
the concentration of the silk polypeptide in the acidic solution is in the range of 0.5% by weight to 40% by weight, more preferably in the range of 5% by weight to 30% by weight, even more preferably in the range of 5% by weight to 25% by weight, and most preferably in the range of 5% by weight to 20% by weight.

It is particularly preferred that the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. It is more particularly preferred that the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In one alternative still even more preferred embodiment, the present invention relates to a method for producing an (a flowable or non-flowable/solid) acidic formulation comprising the steps of:
(i) providing an acidic solution comprising a silk polypeptide and formic acid, wherein the concentration of the silk polypeptide in the solution is in the range of 0.5% by weight to 45% by weight, and wherein the concentration of the formic acid in the solution is in the range of 10% by weight to 90% by weight,
(ii) forming an (flowable or non-flowable/solid) acidic formulation from the solution preferably by setting a pH which is equal or higher than the pI of the silk polypeptide, wherein the pH is set by adding a basic buffer or a base to the solution, and wherein the silk polypeptide is comprised in the (flowable or non-flowable/solid) acidic formulation in an aggregated form and/or has a fibrillary structure, and
(iii) adding a compound (useful in the agrochemical industry, cleaning industry, home care industry, detergent industry, cosmetic industry, or food industry) to the (flowable or non-flowable/solid) acidic formulation.

In particular, the pH is at least 0.1 pH units higher than the pI of the silk polypeptide, e.g. the pH is at least 0.2, 0.3, 0.4, 0.5, 0.6, 0.7, 0.8, 0.9, 1, 2, 3, 4, 5, 6, 7, or 8 pH units higher than the pI of the silk polypeptide.

Preferably,
the concentration of the formic acid in the acidic solution is in the range of 10% by weight to 80% by weight, more preferably in the range of 20% by weight to 75% by weight, even more preferably in the range of 20% by weight to 70% by weight, and most preferably in the range of 25% by weight to 70% by weight, and/or
the concentration of the silk polypeptide in the acidic solution is in the range of 0.5% by weight to 40% by weight, more preferably in the range of 5% by weight to 30% by weight, even more preferably in the range of 5% by weight to 25% by weight, and most preferably in the range of 5% by weight to 20% by weight.

It is particularly preferred that the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight or 40% by weight. It is more particularly preferred that the concentration of the silk polypeptide in the acidic solution is at least 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39% by weight and not more than 40% by weight.

In the above embodiments, silk polypeptide is specifically selected from the group consisting of (C)ₘ, (C^{Cys})ₘ, (C^{Lys})ₘ, (C)ₘC^{Cys}, C^{Cys}(C)ₘ, C^{Lys}(C)ₘ, and (C)ₘC^{Lys}, wherein m is an integer of 2 to 96, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96. For example, the silk polypeptide is selected from the group consisting of C^{Lys}C₁₆, C^{Lys}C₃₂, C^{Lys}C₄₈, C₁₆C^{Lys}, C₃₂C^{Lys}, C₄₈C^{Lys}, C₁₆, C₃₂, C₄₈, C^{Cys}C₁₆, C^{Cys}C₃₂, C^{Cys}C₈, C₁₆C^{Cys}, C₃₂C^{Cys}, and C₄₈C^{Cys}.

In a further step, the (flowable or non-flowable/solid) acidic formulation produced in the method of the second aspect can be applied to a substrate or added to/incorporated into/worked into a composition. In other words, the (flowable or non-flowable/solid) acidic formulation produced in the method of the second aspect can be coated onto a substrate or added to/incorporated/worked into a composition.

It is particularly preferred that the (flowable or non-flowable/solid) acidic formulation, e.g. hydrogel, of the first aspect is obtainable by the method of the second aspect. It is particularly more preferred that the (flowable or non-flowable/solid) acidic formulation, e.g. hydrogel, of the first aspect, is produced by the method of the second aspect.

It is assumed that formic acid, in particular the produced formate, stabilizes the silk polypeptides in the (flowable or non-flowable/solid) acidic formulation. Thus, in a third aspect, the present invention relates to a (flowable or non-flowable/solid) acidic formulation of the first aspect for silk polypeptide storage. Preferably, the (flowable or non-flowable/solid) acidic formulation has a neutral pH, e.g. a pH of 7±0.5

The (flowable or non-flowable/solid) acidic formulation of the first aspect and produced according to the method of the second aspect can be used universally for all applications. In contrast to the previous production process for silk solutions, expensive solvents and complex procedures can be dispensed with. Thus, the manufacturing costs can be significantly reduced. Thus, in a fourth aspect, the present invention relates to the use of the (flowable or non-flowable/solid) acidic formulation of the first aspect in the agrochemical industry, cleaning industry, home care industry, detergent industry, cosmetic industry, or food industry, preferably pet food industry. For example, the (flowable or non-flowable/solid) acidic formulation of the first aspect can be added to agrochemical products such as fertilizers or plant protection products. In particular, agrochemical products can be coated with the (flowable or non-flowable/solid) acidic formulation of the first aspect. Alternatively, the (flowable or non-flowable/solid) acidic formulation of the first aspect can be added to foodstuffs or food supplements. In particular, foodstuffs or food supplements can be coated with the (flowable or non-flowable/solid) acidic formulation of the first aspect. The (flowable or non-flowable/solid) acidic formulation can also be added to/incorporated/worked into products used in the agrochemical industry, cleaning industry, home care industry, detergent industry, cosmetic industry, or food industry.

In a fifth aspect, the present invention relates to a substrate comprising the (flowable or non-flowable/solid) acidic formulation of the first aspect. The substrate may be any substrate that benefits from the (flowable or non-flowable/solid) acidic formulation. It is preferred that the substrate is coated with the (flowable or non-flowable/solid) acidic formulation of the first aspect. The coating is a covering that is applied to the substrate, in particular to the surface of the substrate, to be coated. The coating itself may be an all-over coating, completely covering the substrate, or it may only cover parts of the substrate.

In one embodiment, the coating covers at least 1%, preferably at least 30%, more preferably at least 50%, even more preferably at least 80%, and most preferably at least 90% or even 100% of the surface of the substrate. In one preferred embodiment, the coating is an uniform and/or homogenous coating. It has preferably a thickness of between 10 nm and 1 mm and more preferably a thickness of between 50 nm and 0.5 μm.

The coating has preferably the form of a hydrogel. It is preferably achieved by dip coating and/or spray coating.

In an example, the substrate is a plant. In this case, the coating may be applied to (parts of) the root(s) of the plant and/or sprout(s) of the plant/aboveground part(s) of the plant. The coating may also be applied to (parts of) the fruits and/or blossoms/flower petals of the plant. It should be clear that a plant, in particular (a/an) sprout(s) of a plant/aboveground part(s) of a plant, also comprise(s) leaves/leafage. Thus, the coating preferably also covers the leaves/leafage. The coating is preferably applied pre-harvest, e.g. to a seedling, a growing plant, or full-grown plant.

Said substrate may be selected from the group consisting of a plant, a part of a plant (e.g. root and/or sprout), plant seeds, a nutrient, a fruit, and a flower.

In a sixth aspect, the present invention relates to a composition comprising the (flowable or non-flowable/solid) acidic formulation of the first aspect.

The composition may be any composition that benefits from the (flowable or non-flowable/solid) acidic formulation. The composition may be, for example, a composition useful in the agrochemical industry, cleaning industry, home care industry, detergent industry, cosmetic industry, or food industry. Thus, said composition may be a food composition, a cleaning composition, a fertilizer composition, or a plant or seed protecting composition. In these cases, the (flowable or non-flowable/solid) acidic formulation may be integrated, embedded, or worked into the composition.

Various modifications and variations of the invention will be apparent to those skilled in the art without departing from the scope of invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art in the relevant fields are intended to be covered by the present invention.

### BRIEF DESCRIPTION OF THE FIGURE

The following figures are merely illustrative of the present invention and should not be construed to limit the scope of the invention as indicated by the appended claims in any way.

FIG. 1: Solid hydrogel after immediate addition of base. Concentration of silk protein in final formulation is 50 g/L.

FIG. 2: Solid hydrogel after stepwise addition of base. Concentration of silk polypeptide in final formulation is 77 g/L.

FIG. 3: Left: silk polypeptide solution before increase of pH. Right: flowable silk polypeptide formulation with a silk polypeptide concentration of 6.5 g/L and a pH of 4-5. It should be noted that the silk polypeptide which is comprised in the formulation has a fibrillary structure.

FIG. 4: Left: silk polypeptide solution before increase of pH. Right: flowable silk polypeptide formulation with a silk polypeptide concentration of 32.5 g/L and a pH of 4-5. It should be noted that the silk polypeptide which is comprised in the formulation has a fibrillary structure.

### EXAMPLES

The examples given below are for illustrative purposes only and do not limit the invention described above in any way.

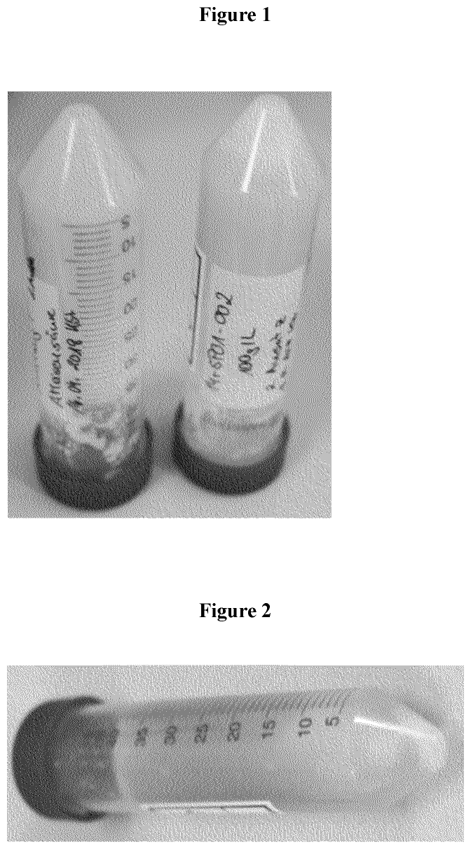

Silk protein was dissolved in formic acid. Subsequently, pH increase with NaOH was performed. This led to a gelation of the solution (see FIG. 1-4).

1. Production of Solid Silk Polypeptide Formulations from Formic Acid

### 1.1 Example 1

For production of a solid silk polypeptide formulation by immediate addition of base, 1.5 g of the silk polypeptide C₁₆ were weighed in a 50 mL reaction tube. The reaction tube was filled with formic acid to a final volume of 15 mL. The resulting concentration of the silk polypeptide C₁₆ was 100 g/L. The protein was solubilized using an overhead shaker for sufficient mixing. After 3 hours of mixing, the protein was solubilized completely.

Sodium hydroxide was used as base for pH correction at a concentration of 10 M. 15 mL of sodium hydroxide were added to the formic acid with solubilized silk protein. A strong temperature increase could be observed. Final concentration of silk polypeptide in the final formulation was 50 g/L.

Surprisingly, the solution gelled immediately. The solid silk polypeptide formulation was stable (see FIG. 1). FIG. 1 shows a solid hydrogel after immediate addition of base. Concentration of silk protein in final formulation is 50 g/L.

### 1.2 Example 2

For production of a solid silk polypeptide formulation by stepwise addition of base, 3 g of the silk polypeptide C₁₆ were weighed in a 50 mL reaction tube. The reaction tube was filled with formic acid to a final volume of 30 mL. The resulting concentration of the silk polypeptide C₁₆ was 100 g/L. The protein was solubilized using an overhead shaker for sufficient mixing. After 3 hours of mixing, the protein was solubilized completely.

Sodium hydroxide was used as base for pH correction at a concentration of 10 M. In total 9 mL of sodium hydroxide were added stepwise (1-2 mL per step) to the formic acid with solubilized silk polypeptide. After each addition, the solution was mixed for at least 1 minute. Final concentration of silk polypeptide in the final formulation was 77 g/L.

Surprisingly, the solution gelled overnight. The solid hydrogel was stable (see FIG. 2). FIG. 2 shows a solid hydrogel after stepwise addition of base. Concentration of silk polypeptide in final formulation is 77 g/L.

2. Production of Flowable Silk Polypeptide Formulations from Formic Acid

### 2.1 Example 3

For production of a flowable silk polypeptide formulation, 0.28 g of the silk polypeptide C₁₆ were weighed in a 50 mL reaction tube. The reaction tube was filled with formic acid to a final volume of 14 mL. The resulting concentration of the silk polypeptide C₁₆ was 20 g/L. The protein was solubilized using an overhead shaker for sufficient mixing. After 3 hours of mixing, the protein was solubilized completely.

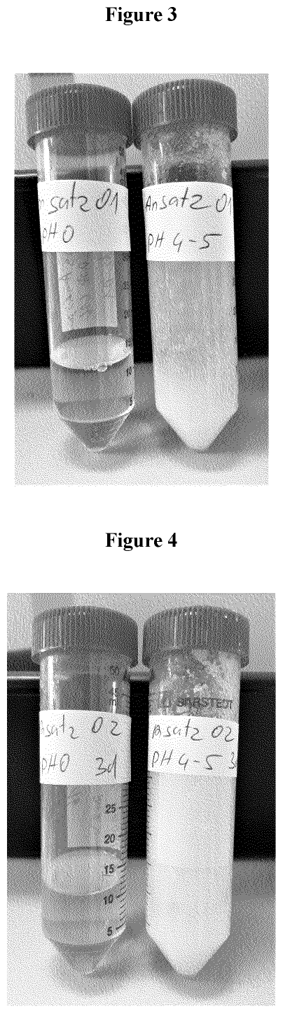

Sodium hydroxide was used as base for pH correction at a concentration of 10 M. In total 26 mL of sodium hydroxide were added stepwise (1-2 mL per step) to the formic acid with solubilized silk polypeptide. After each addition, the solution was mixed for at least 1 minute. The final pH was 4-5. The final silk polypeptide concentration was 6.5 g/L. Surprisingly, the solution turned into a stable, flowable silk polypeptide formulation (see FIG. 3). FIG. 3 shows a silk polypeptide solution before pH increase (left) and a flowable silk polypeptide formulation with a silk polypeptide concentration of 6.5 g/L and a pH of 4-5 (right). It should be noted that the silk polypeptide which is comprised in the formulation has a fibrillary structure.

### 2.2 Example 4

For production of a flowable silk polypeptide formulation by stepwise addition of base, 1.4 g of the silk polypeptide C16 were weighed in a 50 mL reaction tube. The reaction tube was filled with formic acid to a final volume of 14 mL. The resulting concentration of the silk polypeptide C16 was 100 g/L. The protein was solubilized using an overhead shaker for sufficient mixing. After 3 hours of mixing, the protein was solubilized completely.

Sodium hydroxide was used as base for pH correction at a concentration of 10 M. In total 26 mL of sodium hydroxide were added stepwise (1-2 mL per step) to the formic acid with solubilized silk polypeptide. After each addition, the solution was mixed for at least 1 minute. The final pH was 4-5. The final silk polypeptide concentration was 32.5 g/L. Surprisingly, the solution turned into a stable, flowable silk polypeptide formulation (see FIG. 4). FIG. 4 shows a silk polypeptide solution before pH increase (left) and a flowable silk polypeptide formulation with a silk polypeptide concentration of 32.5 g/L and a pH of 4-5 (right). It should be noted that the silk polypeptide which is comprised in the formulation has a fibrillary structure.

1. An acidic formulation comprising a silk polypeptide and formate, wherein the concentration of the silk polypeptide in the formulation is in the range of 0.25% by weight to 45% by weight, and wherein the silk polypeptide is comprised in the formulation in an aggregated form.

2. (canceled)

3. The acidic formulation of claim 1, wherein the silk polypeptide has a fibrillary structure.

4. The acidic formulation of claim 1, wherein the formulation has a gel-like structure.

5. (canceled)

6. The acidic formulation of claim 1, wherein the formulation has a pH which is equal or higher than the pI of the silk polypeptide.

7. The acidic formulation of claim 6, wherein the pH is at least 0.1 pH units higher than the pI of the silk polypeptide.

8. (canceled)

9. The acidic formulation of claim 1, wherein the concentration of the formate in the formulation is in the range of 10% by weight to 90% by weight.

10. The acidic formulation of claim 1, wherein the silk polypeptide comprises at least two identical repetitive units, wherein the repetitive units are independently selected from the group consisting of module C (SEQ ID NO: 1) or variants thereof, module C^{Cys} (SEQ ID NO: 2) or variants thereof, and module C^{Lys} (SEQ ID NO: 7) or variants thereof.

11. (canceled)

12. The acidic formulation of claim 1, wherein the silk polypeptide is selected from the group consisting of (C)ₘ, (C^{Cys})ₘ, (C^{Lys})ₘ (C)ₘC^{Cys}, C^{Cys}(C)ₘ, C^{Lys}(C)ₘ, and (C)ₘC^{Lys}, wherein m is an integer of 2 to 96.

13. (canceled)

14. The acidic formulation of claim 1, wherein the acidic formulation is a flowable acidic formulation, wherein the concentration of the silk polypeptide in the flowable acidic formulation is in the range of 0.25% by weight to 4% by weight.

15. (canceled)

16. The acidic formulation of claim 1, wherein the acidic formulation is a non-flowable/solid acidic formulation, wherein the concentration of the silk polypeptide in the non-flowable/solid acidic formulation is in the range of >4% by weight to 45% by weight.

17. (canceled)

18. A method for producing an acidic formulation comprising the steps of:
(i) providing an acidic solution comprising a silk polypeptide and formic acid, wherein the concentration of the silk polypeptide in the solution is in the range of 0.5% by weight to 45% by weight, and
(ii) forming an acidic formulation from the solution, wherein the silk polypeptide is comprised in the formulation in an aggregated form.

19. (canceled)

20. The method of claim 18, wherein the acidic formulation is formed by setting a pH which is equal or higher than the pI of the silk polypeptide.

21. The method of claim 20, wherein the pH is at least 0.1 pH units higher than the pI of the silk polypeptide.

22. The method of claim 20, wherein the pH is set by adding a basic buffer or a base to the solution.

23. The method of claim 18, wherein the acidic formulation has a gel-like structure.

24-25. (canceled)

26. The method of claim 18, wherein
the concentration of the formic acid in the solution is in the range of 10% by weight to 90% by weight.

27. The method of claim 18, wherein the silk polypeptide comprises at least two identical repetitive units, wherein the repetitive units are independently selected from the group consisting of module C (SEQ ID NO: 1) or variants thereof, module C^{Cys} (SEQ ID NO: 2) or variants thereof, and module C^{Lys} (SEQ ID NO: 7) or variants thereof.

28. (canceled)

29. The method of claim 18, wherein the silk polypeptide is selected from the group consisting of (C)ₘ, (C^{Cys})ₘ, (C^{Lys})ₘ (C)ₘC^{Cys}, C^{Cys}(C)ₘ, C^{Lys}(C)ₘ, and (C)ₘC^{Lys}, wherein m is an integer of 82 to 96.

30-31. (canceled)

32. The method of claim 18, wherein the acidic formulation in step (ii) is a flowable acidic formulation, formed by the steps of:
(i) providing an acidic solution comprising a silk polypeptide and formic acid, wherein the concentration of the silk polypeptide in the solution is in the range of 0.5% by weight to 4% by weight, and
(ii) forming a flowable acidic formulation from the solution, wherein the silk polypeptide is comprised in the formulation in an aggregated form.

33-34. (canceled)

35. The method of claim 18, wherein the acidic formulation in step (ii) is a non-flowable/solid acidic formulation formed by the steps of:
(i) providing an acidic solution comprising a silk polypeptide and formic acid, wherein the concentration of the silk polypeptide in the solution is in the range of >4% by weight to 45% by weight, and
(ii) forming a non-flowable/solid acidic formulation from the solution, wherein the silk polypeptide is comprised in the formulation in an aggregated form.

36-38. (canceled)

39. A substrate or composition comprising the acidic formulation of claim 1.

40. (canceled)
